(19) United States
(12) Patent Application Publication
(10) Pub. No.: US 2024/0325196 A1
(43) Pub. Date: Oct. 3, 2024

(54) HAND OPERATED INTRAOCULAR FLUID DELIVERY DEVICE

(71) Applicant: Nathaniel R. Collins, Monrovia, CA (US)

(72) Inventor: Nathaniel R. Collins, Monrovia, CA (US)

(21) Appl. No.: 18/737,502

(22) Filed: Jun. 7, 2024

Related U.S. Application Data
(63) Continuation-in-part of application No. 18/489,636, filed on Oct. 18, 2023, now abandoned.
(60) Provisional application No. 63/417,633, filed on Oct. 19, 2022.

(51) Int. Cl. A61F 9/00

(52) U.S. Cl. CPC A61F 9/0017

(57) ABSTRACT

Fluid delivery devices and methods for the treatment of ocular disorders requiring targeted and controlled administration of a fluid to an interior portion of the eye for reduction or prevention of symptoms of the disorder. The device can include a surgical instrument coupled to the distal end for an ophthalmic procedure and can have a lumen configured to convey the substance into an intraocular site of a patient. A fluid compartment can be internal to the device or coupled to the device and configured to hold a substance. A rotary pump mechanism in the device can be configured to transfer fluid about its axis to draw the substance from the fluid compartment and eject the substance through the lumen of the tool. A push button or roller wheel can be on the side of the housing and configured to engage the rotary pump to actuate a forward fluid flow towards the distal end of the device. The chamber may house continuous tubing with a rotor within the tubing creating peristaltic fluid motion, or the chamber may be sealed and fluid-filled, with a rotor acting as an impeller to move fluid to the distal end and surgical instrument.

### RELATED APPLICATIONS

The present application is a continuation-in-part of prior U.S. Ser. No. 18/489,636, filed Oct. 18, 2023 which claims priority to prior U.S. provisional Ser. No. 63/417,633, filed Oct. 19, 2022.

### FIELD OF THE INVENTION

The present application is directed to a hand-operated intraocular fluid delivery device.

### BACKGROUND OF THE INVENTION

Minimally invasive surgical procedures involving ocular incisions and/or intraocular fluid injection can be useful for treating glaucoma and other eye conditions. For example, in a Trabeculectomy, a surgeon can use an ophthalmic blade inserted through an incision in the eye to remove a portion of the trabecular meshwork, thereby improving outflow of the aqueous humour (AH) and relieving intraocular pressure contributing to glaucoma.

During the removal of the trabecular meshwork using an ophthalmic blade, some cases have been observed in which minor bleeding occurs. When bleeding occurs during the surgery, the blood can cover up the trabecular meshwork and Schlemm's canal, creating a visual obstruction. One method of dealing with the blood reflux involves removing the ophthalmic blade from the eye and inserting a viscoelastic syringe. The viscoelastic syringe can be used to push visco elastic into Schlemm's canal and/or into the collector channels or move blood away from the trabecular meshwork. Once the blood is pushed away from the trabecular meshwork, the viscoelastic syringe is removed from the eye and ophthalmic blade is re-inserted to continue the surgery.

With ophthalmic surgeries and drug deliveries in the eye, there is a need for extreme delicacy and precision by the doctor. Any advantage that a medical device can offer in terms of useability, control and precision of movements has a significant impact on surgical outcomes. Using a syringe to dispense fluid in these procedures is proving inadequate and many doctors and medical device companies have tried to find alternate means.

There are a handful of ophthalmic devices that have come on the market which have attempted to put a pump like mechanism into a handheld device. Some include pushing fluid by a plunger, while others use a reciprocating pump. Devices having microcannulas or orifices configured to inject a substance into Schlemm's canal or other intraocular sites are disclosed in U.S. Pat. No. 10,722,397 and U.S. Pat. No. 10,946,145. Current designs have drawbacks due to entrapping air in the system, fluid seeping which leads to unintentional discharge, and inadequacy dealing with the high pressures often experienced.

### SUMMARY OF THE INVENTION

This application presents fluid delivery devices and methods for the treatment of ocular disorders requiring targeted and controlled administration of a fluid into the eye for reduction or prevention of symptoms of the disorder.

A commonly used fluid in ophthalmic surgeries is viscoelastic. This fluid is nonnewtonian and extremely viscous. The characteristics of viscoelastic when used through a reciprocating pump are the cause of poor performance in both device output, as well as the high user force required to dispense fluid. A peristaltic design pump, or externally deforming and advancing fluid through a flexible tube, is superior for dispensing these types of fluids. The fluid path can be a continuous flexible tube which eliminates air entrapment and is better for advancing even the most viscous fluids through the system.

A peristaltic pump system contained in a handheld device as disclosed herein will provide the control and precision the doctor requires without the performance limitations of other competing devices. A benefit of a peristaltic pump, like a rotary pump, is that fluid can be drawn out of a reservoir. This reservoir can be an internal chamber, or an externally attached chamber, such as a syringe.

A fluid injection device can include a surgical instrument coupled to the distal end for an ophthalmic procedure and can have a lumen configured to convey the substance into an intraocular site of a patient. A fluid compartment can be internal to the device or coupled to the device and configured to hold a substance.

The device can also have a connector on the distal end, such as a luer connection, to allow for a wide range of attachments.

A fluid compartment can be internal to the device or coupled to the device and configured to hold a substance.

A rotary pump mechanism in the device can be configured to transfer fluid about its axis to draw the substance from the fluid compartment and eject the substance out the front of the tool.

A push button or rolling finger wheel can be on the side of the housing and configured to engage the rotary pump to actuate a forward fluid flow towards the distal end of the device. Alternatively, reverse flow or aspiration can also be possible to use the system as a vacuum to remove substances from the eye. Any manually-operated button, wheel, lever, roller, slider, dial, etc. may be adapted operate the internal fluid pump from outside the housing and cause fluid to flow out the distal cannula. Therefore, the term “manual actuator” encompasses all of these possible fluid-drive mechanisms.

Pressing the handle or rolling the finger wheel causes the mechanism to engage, causing the rollers to crush/squeeze the tubing (Durometer Shore80A or less) which moves the fluid though the tubing and into the dispensing tip to be delivered to the patient. Specifically, this system works by externally compressing the tube and creates the sliding motion by rotating the wheel to advance the fluid inside the tube. During the process, the motion creates negative pressure inside the tube, drawing the subsequence volume of the fluid to filled up the space previously occupied by the volume of the fluid that has been advanced. This method creates no voids or air bubbles inside the tube and allows accurate control of the fluid volume dispensing out of the tube.

Upon initial use, the handle can be pressed enough times so that fluid is dispensed from the distal end prior to use on the patient. Alternatively, the rollers/arm can be retracted so that the operator can fill the tubing (i.e., prime) the device prior to engaging the handle. Retractable rollers also aid in inserting a replaceable tubing component.

A ratchet, clutch, and one-way bearing may be utilized to prevent the mechanism from reversing direction which reduces risk to the patient. The nature of a manually powered rotary pump also lowers the risk of delivering excess fluid to the patient because there is little pressure buildup within the fluid as compared with other standard fluid pumps.

No part of the mechanism makes contact with the fluid itself. Theoretically, this device could achieve the lowest biocompatibility risk of any ophthalmic pump since the fluid only needs to contact the lumen/flexible tubing and the cannula. However, more components may be necessary in the fluid path for the tubing-to-cannula connection and the tubing-to-fluid compartment connection.

A ratchet, clutch, and one-way bearing may be utilized to prevent the mechanism from reversing direction which reduces risk to the patient. The nature of a manually powered rotary pump also lowers the risk of delivering excess fluid to the patient because there is little pressure buildup within the fluid as compared with other standard fluid pumps.

A push button can be on the side of the housing and configured to engage the rotary pump to actuate a forward fluid flow towards the distal end of the device.

### Replaceable Cartridges

The tubing and components in the fluid path can also be configured to be a separate and replaceable component and act as a cartridge. This allows for a reusable handset to help reduce waste. Also, different variations of cartridges can help the operator select the cartridge configuration that is best suited for the operation. For example, a cartridge with different tubing diameter will allow different volume output per actuation. These cartridges can also be separately packages and sterilized.

### Stored Energy

With a direct drive design, as the handle/roller is actuated, the peristaltic pump, which is directly linked, moves a corresponding amount. The speed and length of actuation directly correlates to fluid output.

With a stored energy design, there is a kinetic force driving the pump. When depressing the handle, it acts as a trigger to release a catch and expend some of the kinetic energy to independently rotate the pump.

Stored energy in the form of a spring can come from a wound clock spring supplying a rotary force to the pump. Stored energy in the form of a spring can also come from a linear spring exerting force on a rack which turns a pinion to supply rotary force to the pump. These stored energy methods can come preloaded or can be loaded by the end user. An example of this would be the user winding up the clock spring or “cocking” the linear spring.

The catch mechanism could be preset or configurable by the user. This catch mechanism would allow escapement of the rotary pump while it has a rotary force applied on it. An example of this type of escapement mechanism with a rotary force applied to it is a clock. In this pump mechanism, a gear with set tooth spacing can dictate the amount of rotation per handle depression. This would allow the user to dispense a set amount of fluid with each handle depression.

This method of stored energy to drive the pump will allow a shorter handle motion along with less force. This is critical for device stability and accuracy when dispensing fluid into the eye.

While the catch mechanism is ideal for dispensing a set amount of fluid, sometimes a continuous flow is desired. When a surgeon uses a syringe to inject fluid, they often dose according to visual feedback. For example, when filling the eye with viscoelastic, they would supply enough fluid to displace all present eye fluid, remove bubbles and hold the shape of the anterior chamber based off their visual feedback.

In place of a catch mechanism, a braking mechanism can be used to supply continuous flow. When the handle is depressed, the brake is released, allowing the pump to rotate by the stored energy of a spring. A braking mechanism could directly constrain the axis of rotation of the pump, or it can engage a moving rack. The fluid flow path after the pump could also be pinched or contain an open/shut valve that the handle controls.

In this design, dampening can be used to set the fluid flow velocity. Dampening will allow the pump to spin at a set speed when released, rather than spin uncontrolled until the stored energy is exhausted. The mechanical drive can be dampened directly or the fluid outflow can be constricted to limit fluid flow exiting the device. An example of controlling the fluid flow rate directly would be a valve that the user can adjust to get the desired flow rate.

### CO2 Design

Another variation of the rack and pinion method to supply rotational force, would be to replace the linear spring with a piston driven by compressed gas. An example of a compressed gas would be a CO2 cartridge that is already commonly sold.

CO2 cartridges are currently used to power a number of mechanical devices. Some examples include parachute deployment devices, air powered rifles, and intraocular lens deployment devices. In these cases, the gas excerpts a force on a mechanical element. With a CO2 cartridge, the CO2 is a liquid while in its container. As the liquid CO2 enters a larger space, such as a cylinder, it turns to gas. While in the expansion phase from liquid to gas, the pressure exerted from the CO2 is very constant. This constant force can be applied to a piston, so that over the length of the stroke of the piston, the force applied to the piston is very constant. A CO2 driven piston driving a rack would have a more uniform force over the length of travel as compared to a spring driving a rack, since the spring force decreases as the spring approaches relaxed length.

### Turbo Design

Another method of supplying rotary force to the peristaltic pump with CO2 is to use a turbocharger like design. In this design, there is a turbine section that is connected mechanically, such as a shaft, to the rotary pump. As the CO2 passes through the turbine or impeller section, it will cause this impeller to rotate and in turn drive the rotary pump that it is coupled to. This design can accommodate either the catch mechanism or braking mechanism to control the fluid outflow.

### Activating the CO2 Cartridge

With a CO2 powered device, the user could activate or charge the device by penetrating the cartridge. As with current CO2 devices on the market, this activation could be accomplished by a twisting, pressing or lever mechanism by the user.

### Electric Motor Drive

Another method of supplying rotary force to the peristaltic pump is with an electric motor. The most widely available industrial peristaltic pumps are commonly driven by an electric motor. In these commercially available designs, the operator either loads tubing into the pump body or connects tubing to inlet and outlet ports on the pump. These pumps are usually large and made to sit on a desk, or be attached to a power tool.

In this design, the peristaltic pump and driving motor are both be scaled down to fit inside of a handset for ophthalmic use. The motor could be driven by an onboard power source (i.e., battery) or coupled to external power. Internalizing these components limits the tubing running out of the device and allows for freedom of movement. It also eliminates the large pump unit that would have to sit in the operating room and possibly inside of the sterile field. By eliminating the external pump unit, the length of travel for the ophthalmic fluid is decreased and therefore reduces waste. Ophthalmic fluid, such as viscoelastic, can be very expensive and often only sold in small vials.

A motor driven pump allows the use of electrical controls mounted on the handset which can be set by the user to dictate the fluid output. The user can select a specific dosage per handle depression or select a continuous flow with adjustable flow rate.

### BRIEF DESCRIPTION OF THE DRAWINGS

Features and advantages of the present invention will become appreciated as the same become better understood with reference to the specification, claims, and appended drawings wherein:

FIG. 1 is a side view of a device for controlled administration of a fluid to an interior portion of the eye having a handle and internal manually-powered rotating peristaltic pump that splits incoming fluid into segments and drives those segments forward to effect fluid transfer from a source of fluid through a cannula mounted at a distal end of the handle;

FIG. 2 is a cross sectional perspective view of FIG. 1 and shows the internal mechanisms and exposed peristaltic roller assembly;

FIG. 3 is a front perspective view of FIG. 2 without an outer housing and with an alternative fluid tubing pathway;

FIG. 4 is an exploded view of FIG. 2;

FIG. 5 is an alternative design to FIG. 2 wherein the distal cannula is a detachable unit, and FIG. 6 is an alternative view thereof;

FIG. 7A is a perspective isolated view of a peristaltic roller assembly which may be used within the hand-held fluid delivery devices herein;

FIG. 7B is an exploded view of FIG. 7A with labeled components;

FIGS. 8A and 8B demonstrate how the mechanism of FIG. 1 rotates the roller assembly when force is applied to the end of an actuation handle;

FIGS. 9A-9C demonstrate how the internal roller assembly of the hand-held fluid delivery devices may advance each time the handle is pressed;

FIG. 10 is an alternative hand-held fluid delivery device similar to FIG. 1 in a cross-sectional perspective view with a spring-biased wiper assembly replacing the peristaltic roller assembly;

FIGS. 11A-11C demonstrate the device of FIG. 10 as the handle is pressed, released, and allowed to reset;

FIG. 12 is an exploded view of the fluid path of the device in FIG. 10 with labeled components;

FIGS. 13A-13C demonstrate how the wiper assembly in the device of Figure may advance each time the handle is pressed;

FIG. 14 is an alternative hand-held fluid delivery device similar to FIG. 1 in a cross-sectional perspective view with a star-shaped impeller having flexible vanes replacing the peristaltic roller assembly;

FIG. 15 is an exploded view of the fluid path of the device in FIG. 14 with labeled components;

FIG. 16 shows the rotational direction of the wiper wheel in FIG. 14 with each spoke representing a stage in rotation;

FIG. 17 demonstrates how a syringe may be attached to the back to introduce fluid to the system;

FIG. 18 is an alternative design of FIG. 17 where fluid is collected into an internal reservoir before dispensing;

FIG. 19 is an alternative hand-held fluid delivery device similar to FIG. 1 with an electric motor drive;

FIG. 20 is a cross sectional perspective view of FIG. 19 and shows the internal mechanisms and electronics assembly;

FIG. 21 is a perspective isolated view of the electronics assembly of FIG. 20;

FIG. 22 is an alternative hand-held fluid delivery device similar to FIG. 20 with replaceable fluid path tubing;

FIG. 23 is a perspective view of the replaceable fluid path tubing inserted into the assembly of FIG. 22;

FIG. 24 is a perspective view of FIG. 23 with side door closed;

FIG. 25 is a perspective isolated view of the replaceable fluid path tubing of FIG. 22;

FIG. 26A-26B are side views of the pre and post tubing loading of FIG. 22;

FIG. 27A demonstrates how the roller assembly may retract to allow tube loading;

FIG. 27B demonstrates how the roller assembly may expand to engage with the tubing;

FIG. 28 is an alternate hand-held fluid delivery device that replaces a handle with a roller wheel;

FIG. 29 is a cross sectional perspective view of FIG. 28 and shows the internal gear mechanisms;

FIG. 30 is a perspective isolated view of the mechanism of FIG. 29;

FIG. 31 is an alternate perspective view of FIG. 31 with the side door open;

FIG. 32 is an alternate design of a hand-held fluid delivery device with 2 tubes for selective irrigation and aspiration;

FIG. 33 is a perspective isolated view of FIG. 32 showing internal components;

FIG. 34 is perspective isolated view of FIG. 32 showing further internal components and labeling;

FIG. 35A is an isolated view of the selector and roller mechanism. The selector is in the forward position which allows the inner rollers to engage and provide irrigation fluid flow;

FIG. 35B is an isolated view of the selector and roller mechanism. The selector is in the middle position which allows the inner and outer rollers to engage and provide irrigation and aspiration fluid flow;

FIG. 35C is an isolated view of the selector and roller mechanism. The selector is in the back position which allows the outer rollers to engage and provide aspiration fluid flow;

FIG. 36 is front perspective view of FIG. 33;

FIG. 37 is an isolated view of the dual tube cartridge; and

FIG. 37A is a close-up of the distal end of FIG. 37 to show dual fluid ports.

### DETAILED DESCRIPTION OF THE PREFERRED EMBODIMENTS

The present application provides fluid delivery devices and methods for the treatment of ocular disorders requiring targeted and controlled administration of a fluid to an interior portion of the eye for reduction or prevention of symptoms of the disorder. Applications may include: injecting visco elastic into the SC, injecting mitomycin under the conjunctiva, and medicine delivery via intravitreal injection.

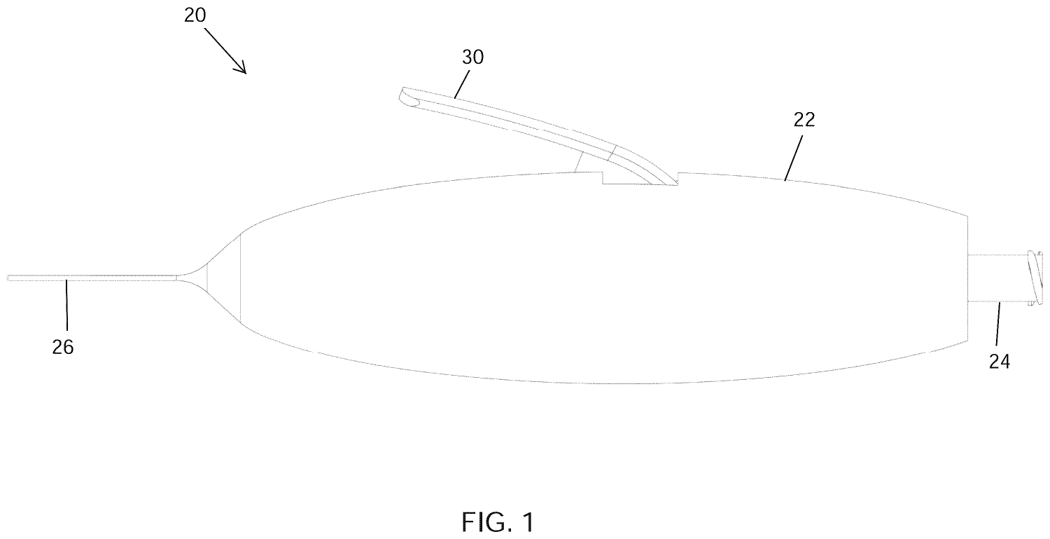

FIG. 1 is a side view of a device 20 for controlled administration of a fluid to an interior portion of the eye having a handle and internal manually-powered rotating peristaltic pump that splits incoming fluid into segments and drives those segments forward to effect fluid transfer from a source of fluid through a cannula mounted at a distal end of the handle.

The device 20 has a hollow outer housing 22 with a proximal luer fitting 24 and a distal surgical instrument 26. The instrument 26 may be a needle, a bent or straight tube, or another specially designed part to interface with selected tissue. In the illustrated embodiment, the instrument 26 is a straight cannula having a distal tip adapted for insertion into an intraocular space in an eye. The device 20 further includes an actuator 30 for advancing fluid in a proximal-distal direction through the device. The outer housing 22 has a generally lenticular shape with convex outer walls that taper down at each end. The housing 22 is sized to be hand-held, and may be between 2-8 inches long and between 0.4-2.5 inches in diameter.

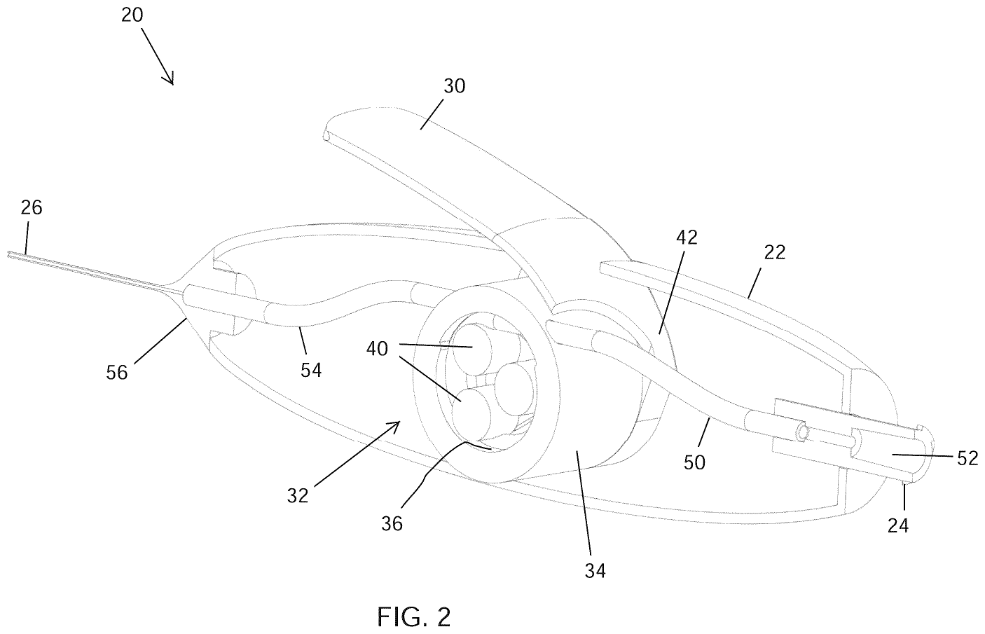

FIG. 2 is a cross sectional perspective view of FIG. 1 and shows the internal mechanisms and an exposed peristaltic pump assembly 32. The assembly 32 includes a hollow chamber 34 mounted within the outer housing 22 and defining an inner wall 36. A plurality, preferably three, of rollers 40 are mounted for rotation within the chamber 34 and form a rotor. The actuator 30 is connected to a lever 42 or other such device which causes the three rollers 40 to rotate.

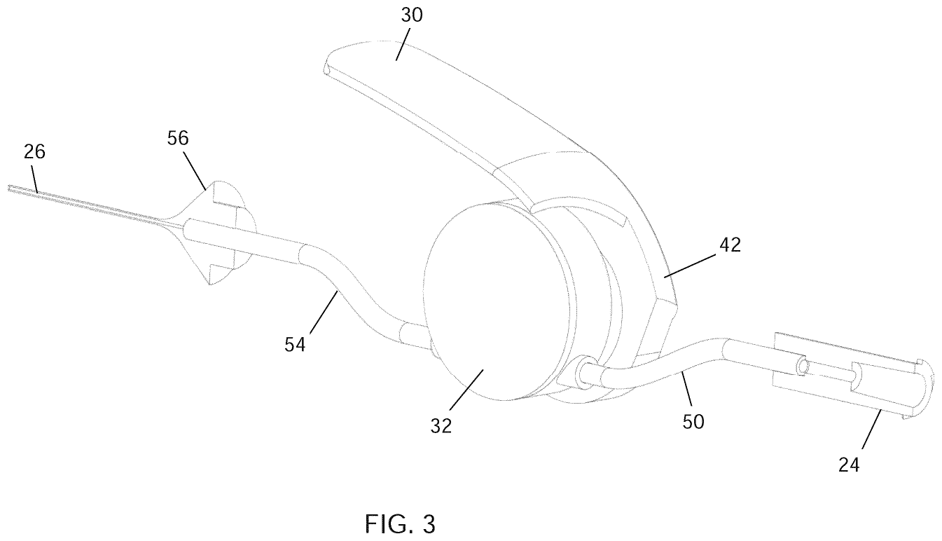

Flexible inlet tubing 50 connects to a fluid inlet 52 defined by the proximal luer fitting 24 and extends to the chamber 34. Flexible outlet tubing 54 connects to a fluid outlet 56 defined by a proximal end of cannula 26 and extends to the chamber 34. The inlet tubing 50 and outlet tubing 54 in the illustrated embodiment are contiguous and define a helical loop 58 around the inner wall 36 of the chamber 34. FIG. 3 shows the fluid component path without the outer housing 22. Rotation of the rollers 40 creates peristaltic movement of fluid within the tubing that extends through the device 20, as will be seen below.

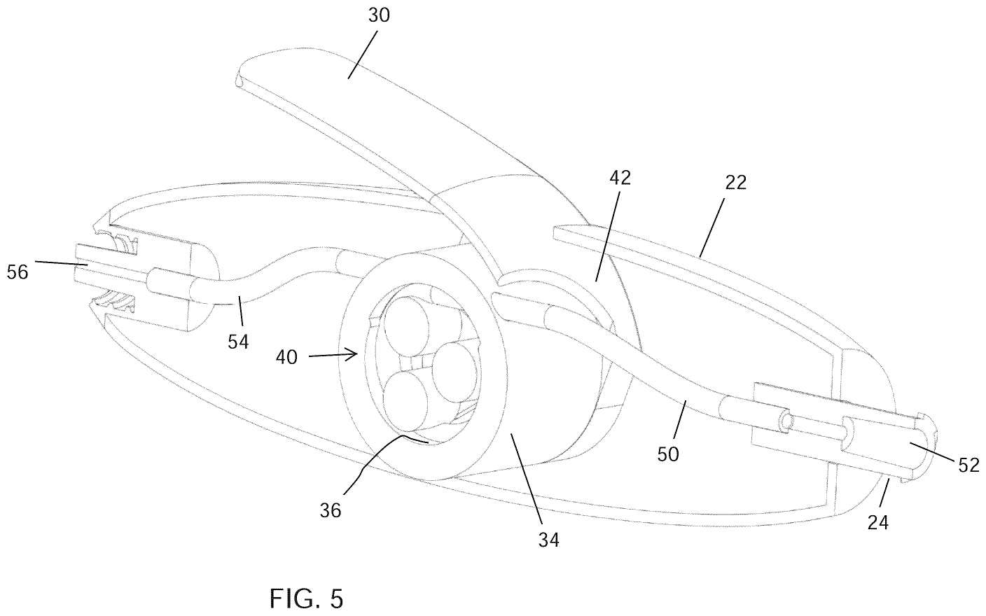

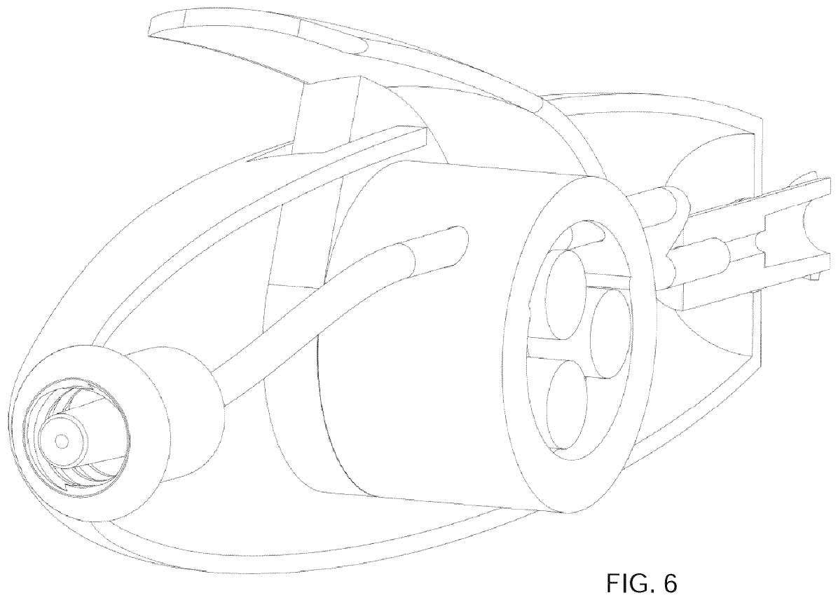

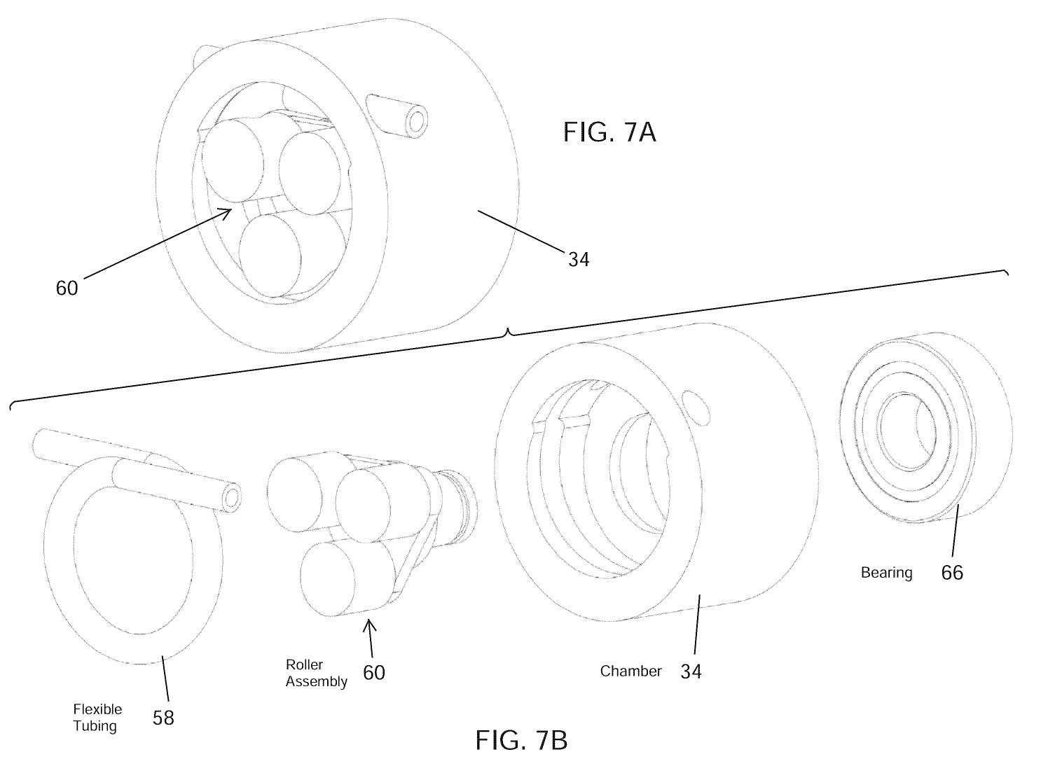

FIG. 5 is an alternative design of the device 20 of FIG. 2 wherein the distal cannula instrument (not shown) is provided as a detachable unit that connects to the fluid outlet 56 by, for example, mating threading, a bayonet lock or a luer lock.

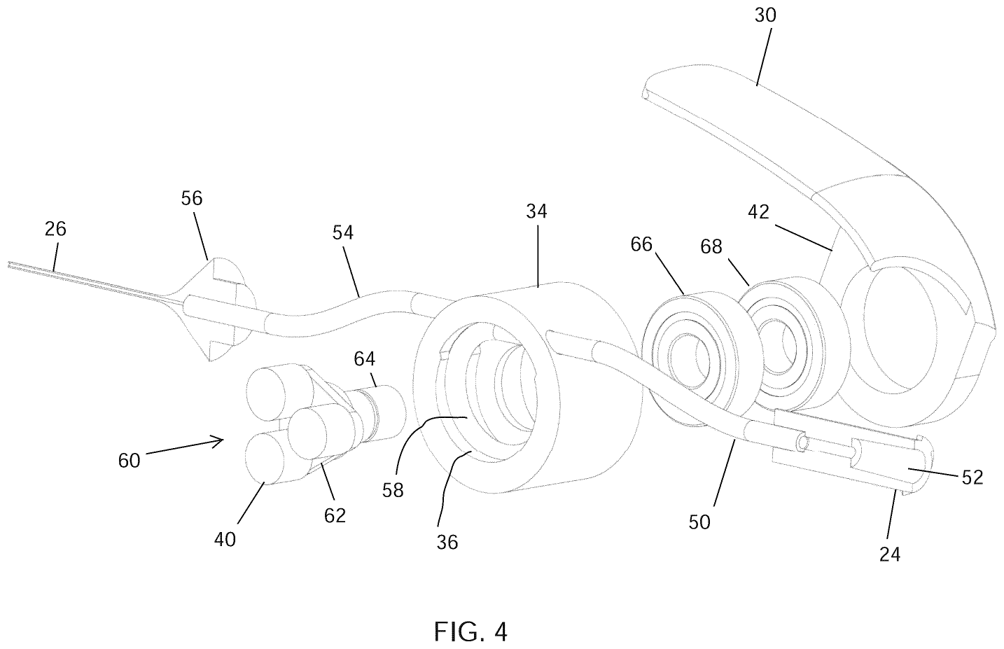

FIG. 4 is an exploded view of FIG. 2 and shows the rotor 60 comprising the three rollers 40 mounted on a common plate 62 and having a shaft stub 64 extending therefrom. The helical loop 58 of tubing is also shown in better detail. When assembled, the rollers 40 are aligned with the helical loop 58 of tubing and the shaft stub 64 extends into a through bore of at least a first bearing 66. The lever 42 of the actuator 30 may be formed in a ring shape that mounts to a second bearing 68. Although not shown, a ratcheting mechanism is provided such that the pressing the actuator 30 rotates the rotor 60 in only one direction. That is, mating teeth or a pawl and tooth assembly is provided between the lever 42 and the rotor 60 such that the pressing the actuator 30 rotates the rotor, while releasing the actuator allows the mechanism to return to its original position without reversing the rotation of the rotor.

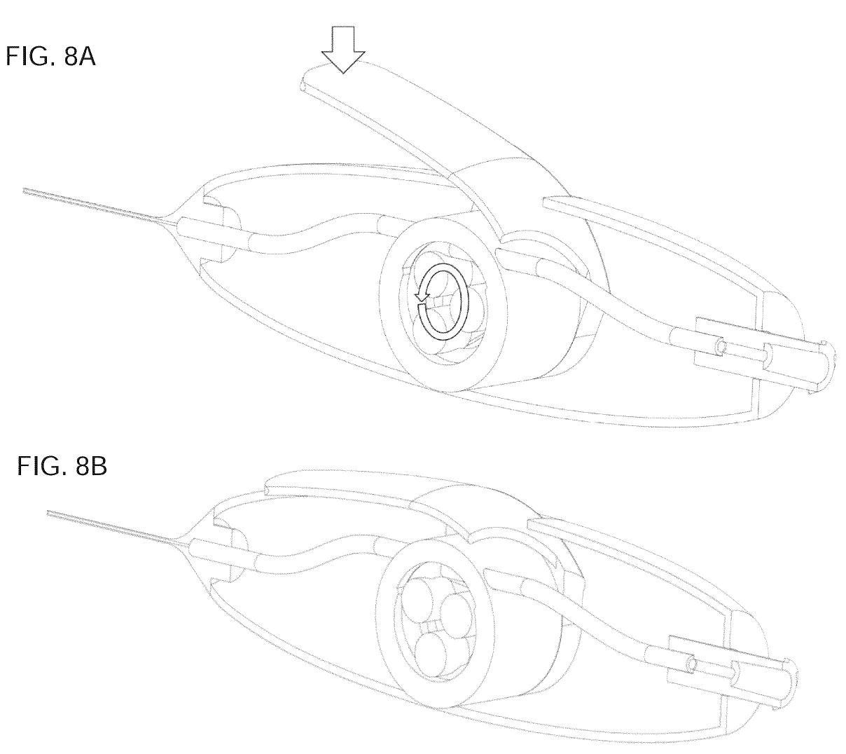

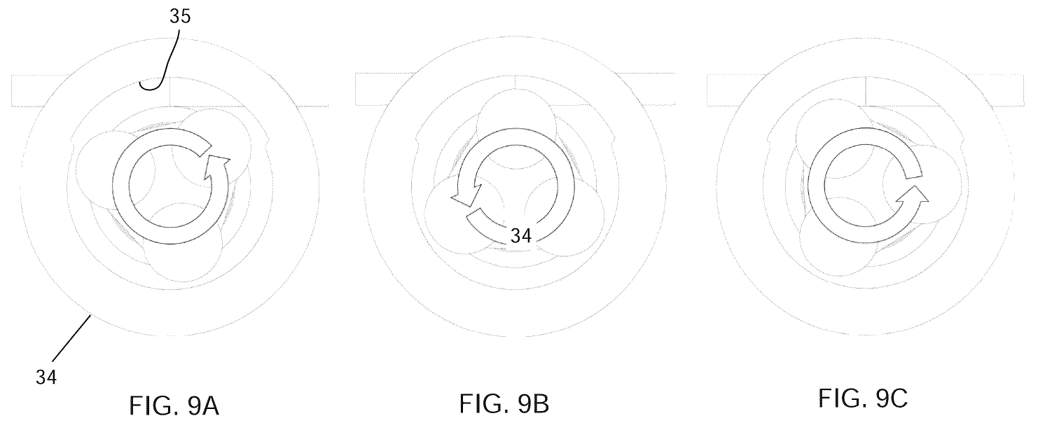

FIGS. 8A and 8B demonstrate how the mechanism of FIG. 1 rotates the roller assembly when force is applied to the end of the handle, and FIGS. 9A-9C demonstrate how the roller assembly may advance each time the handle is pressed. An inner wall of the chamber 34 has a relief portion 35 that is radially larger than the rest of the inner cavity. This relief portion 35 helps the rotor 60 draw fluid into and propel fluid through the tubing 58.

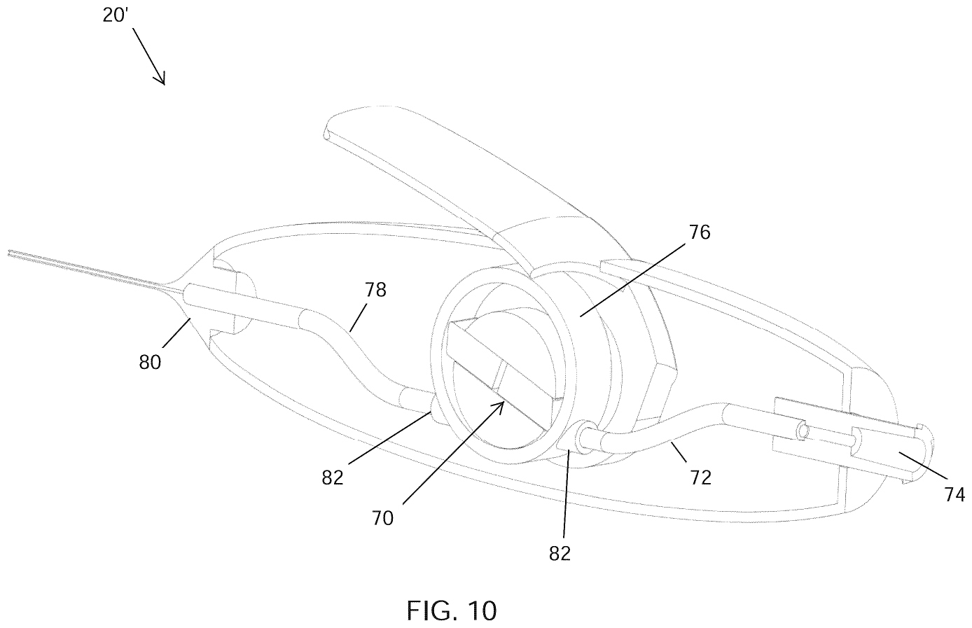

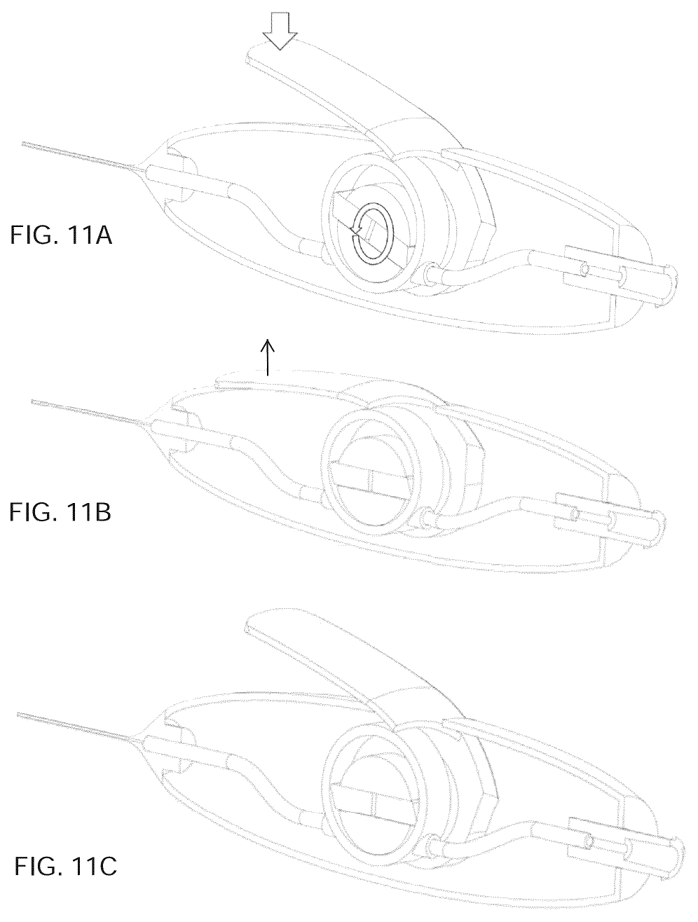

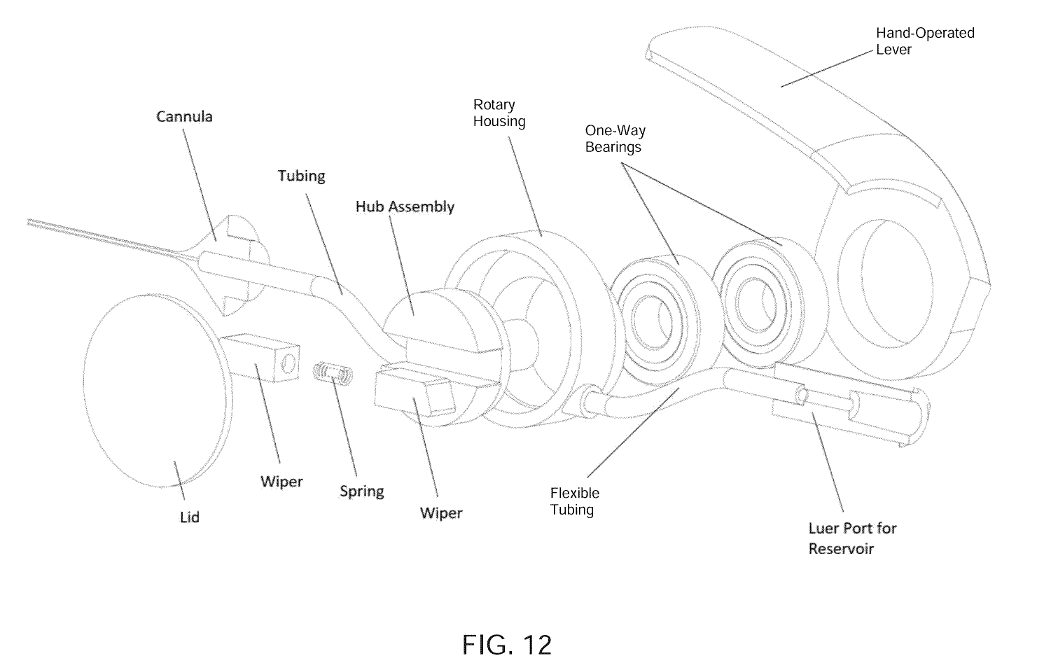

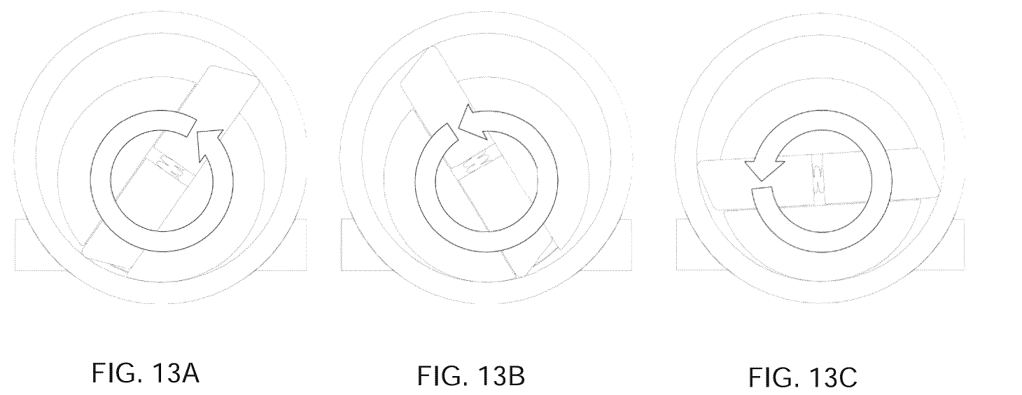

FIG. 10 is an alternative design of a device 20′ such as in FIG. 1 in a cross-sectional perspective view with a spring wiper assembly 70 replacing the rotor assembly 60. FIGS. 11A-11C demonstrate how the wiper assembly 70 may advance each time the handle is pressed, released, and allowed to reset. FIG. 12 is an exploded view of the fluid path of the device in FIG. 10 with labeled components. FIGS. 13A-13C demonstrate how the wiper assembly 70 may advance each time the handle is pressed.

In the embodiment of FIGS. 10-13, flexible inlet tubing 72 connects to the fluid inlet 74 and extends to the chamber 76, while flexible outlet tubing 78 connects to the fluid outlet 80 and extends to the chamber. Both inlet and outlet tubing 72, 78 terminate at fittings 82 extending through the wall of the chamber 76. The interior of the chamber 76 is sealed such that fluid may be provided through the inlet tubing 72 to fill the chamber. The rotor directly contacts the fluid and impels the fluid to move through the chamber to the outlet tubing 78.

Namely, the spring wiper assembly 70 includes a pair of solid wipers that are aligned within a linear channel in an off-center hub assembly and biased apart by an inner spring. As the off-center hub assembly rotates within the chamber 76, the wipers slide in and out of the channel as seen in FIGS. 13A-13B to alternately collect and dispense fluid. The outer ends of the wipers are shaped to “wipe” fluid from the inner wall of the chamber 76 and propel it rotationally. As the wiper assembly 70 rotates in one direction, the wipers therein propel fluid in a proximal-distal direction through the device 20′. All other aspects and options are similar to those described above with respect to the first embodiment.

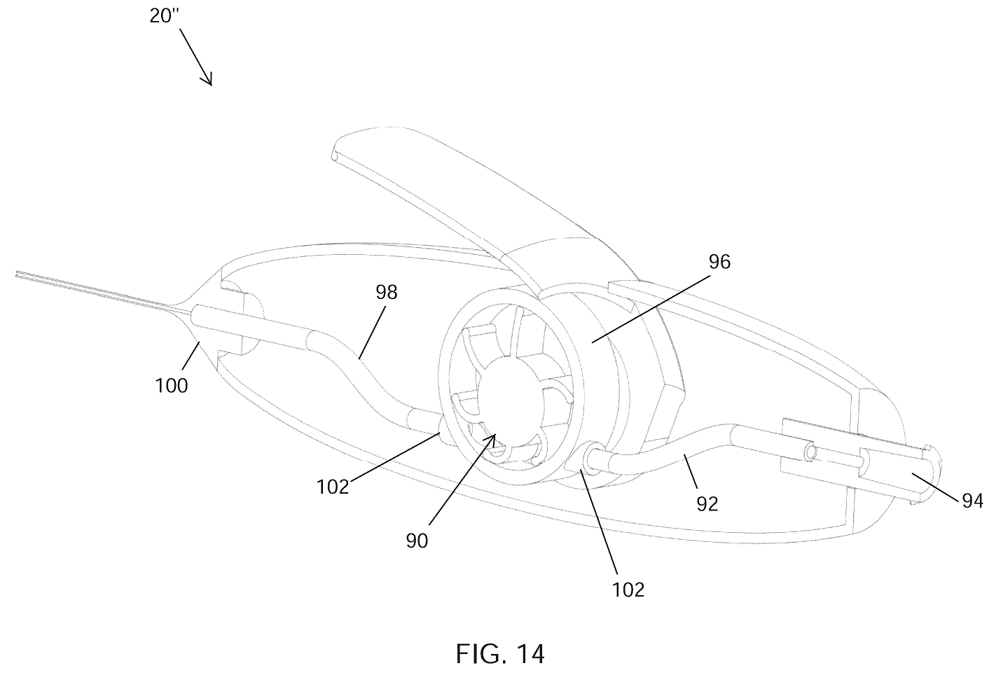

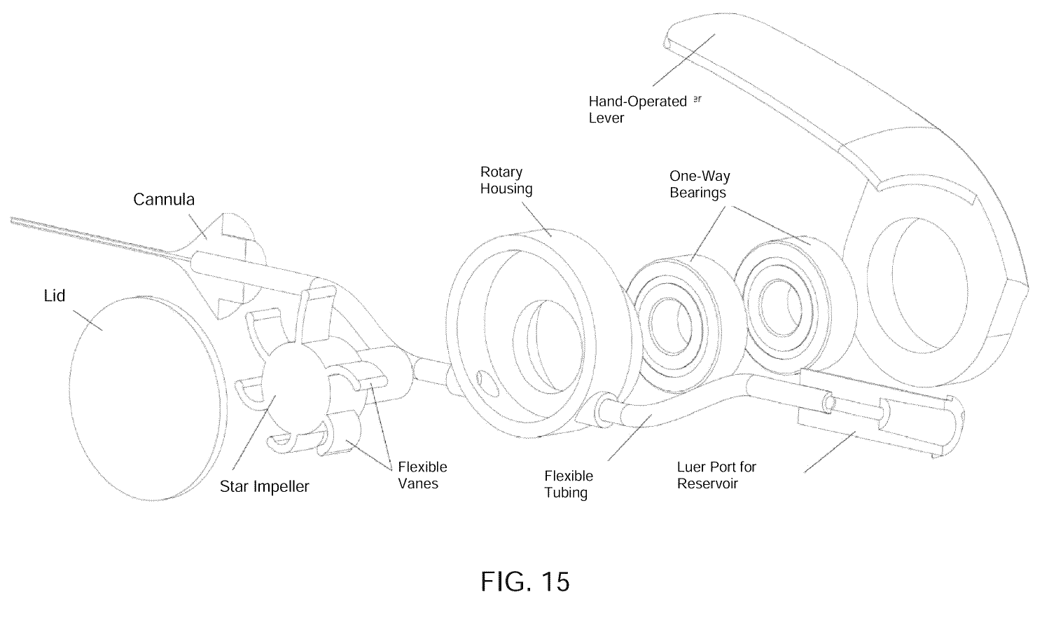

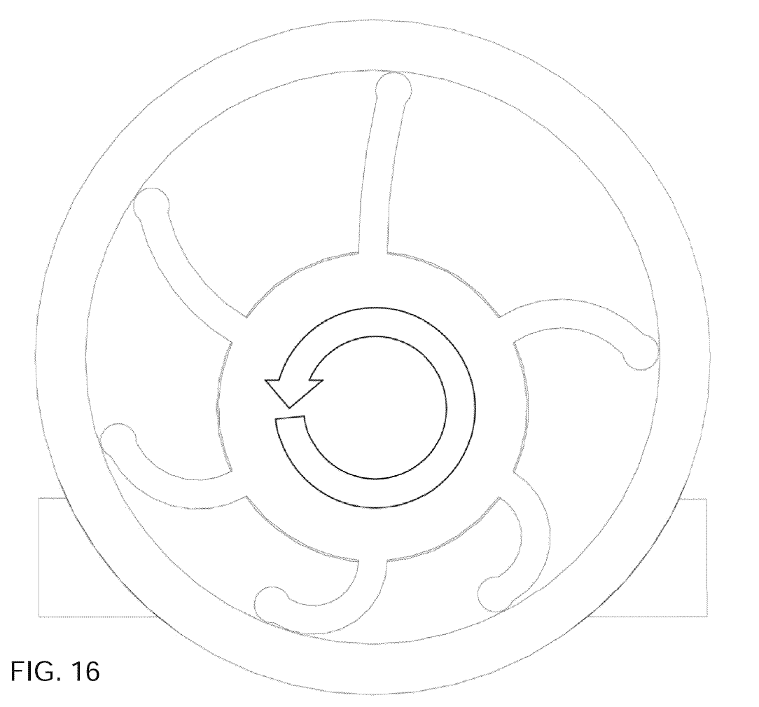

FIG. 14 is a second alternative of a device 20″ such as in FIG. 1 in a cross-sectional perspective view with a star-shaped impeller 90 with flexible spokes or vanes replacing the roller assembly. FIG. 15 is an exploded view of the fluid path of the device in FIG. 14 with labeled components. FIG. 16 shows the rotational direction of the wiper wheel 90 with each spoke representing a stage in rotation.

In the embodiment of FIGS. 14-16, flexible inlet tubing 92 connects to the fluid inlet 94 and extends to the chamber 96, while flexible outlet tubing 98 connects to the fluid outlet 100 and extends to the chamber. Both inlet and outlet tubing 92, 98 terminate at fittings 102 extending through the wall of the chamber 96. The interior of the chamber 96 is sealed such that fluid may be provided through the inlet tubing 92 to fill the chamber. The rotor directly contacts the fluid and impels the fluid to move through the chamber to the outlet tubing 98.

Namely, the star-shaped impeller 90 includes a number of flexible spokes or vanes of the same length projecting outward from an off-center hub that rotates within the chamber 96. Because of the off-center hub, the flexible spokes or vanes alternately collect and dispense fluid as seen in FIG. 16. The outer ends of the wipers bend and “wipe” fluid from the inner wall of the chamber 96 and propel it rotationally. As the wiper assembly 90 rotates in one direction, the wipers therein propel fluid in a proximal-distal direction through the device 20″. All other aspects and options are similar to those described above with respect to the first embodiment.

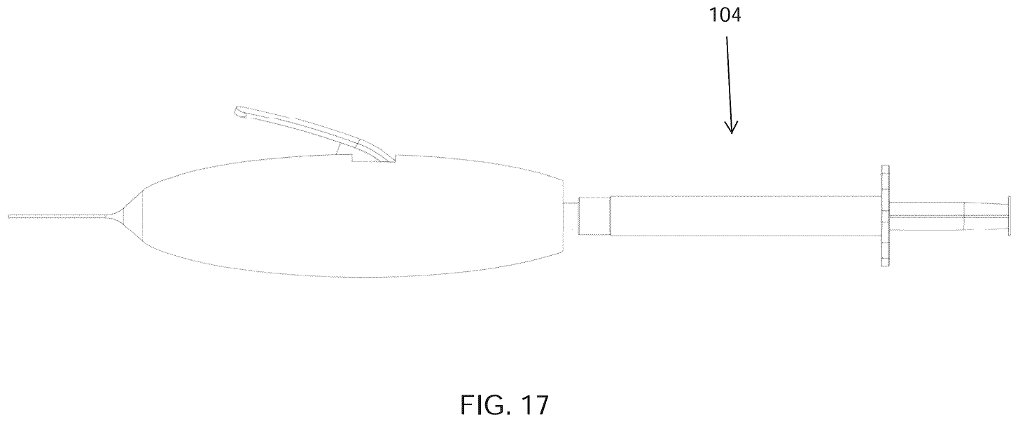

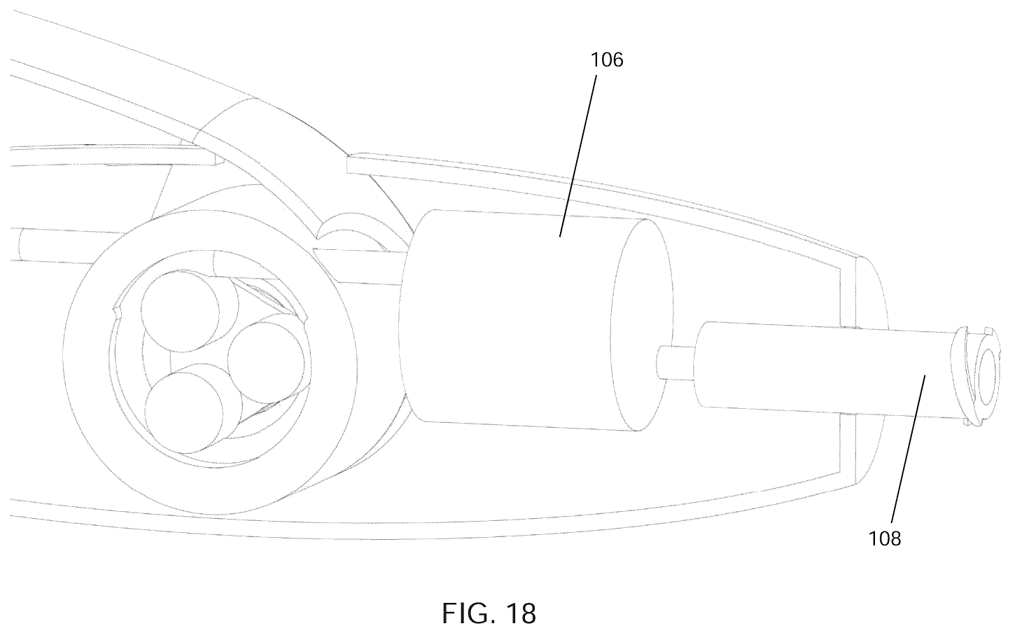

FIG. 17 demonstrates how a syringe 104 may be attached to the back to introduce fluid to the system. Fluid may be injected using the syringe 104 to fill internal chambers in the device, and then the handle can propel the fluid in a controlled manner as described. FIG. 18 is an alternative design of FIG. 17 where fluid is collected into an internal reservoir 106 before dispensing. A luer fitting 108 to which the syringe 104 attaches may be capped off with a one-way valve cap to permit the handle to propel the fluid in a controlled manner from the reservoir 106.

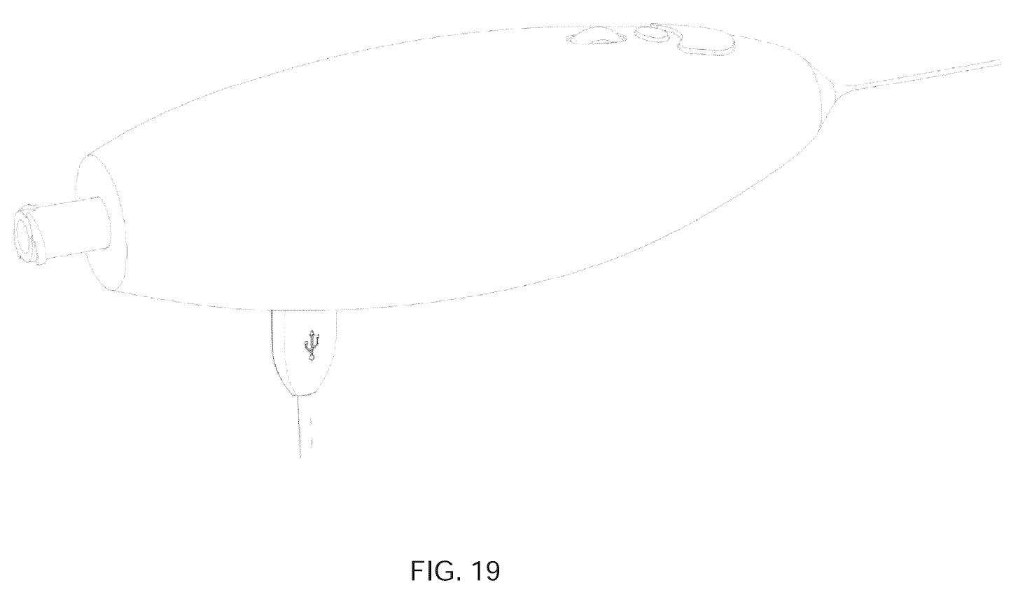

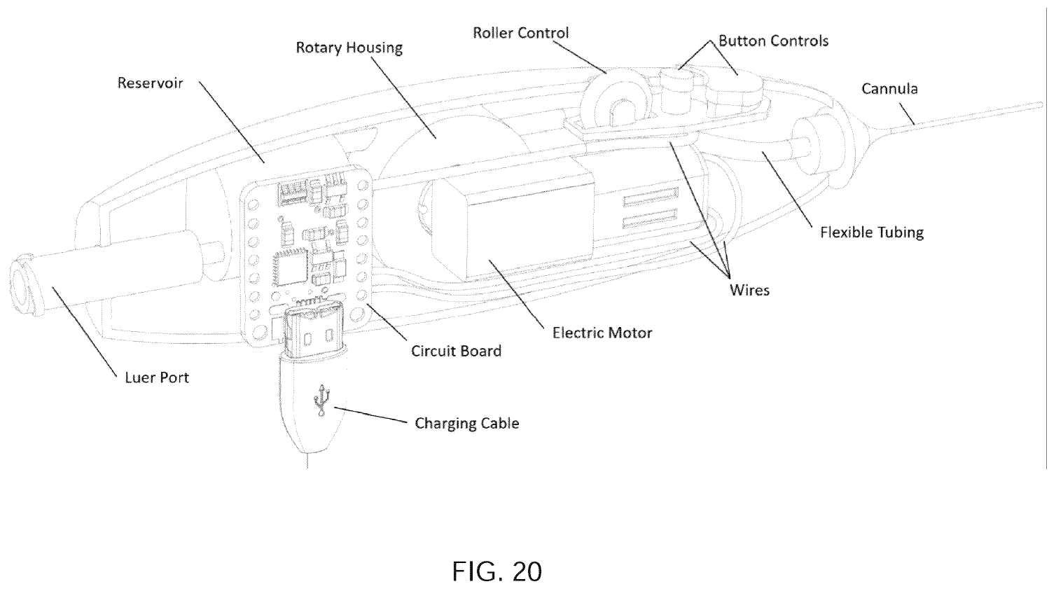

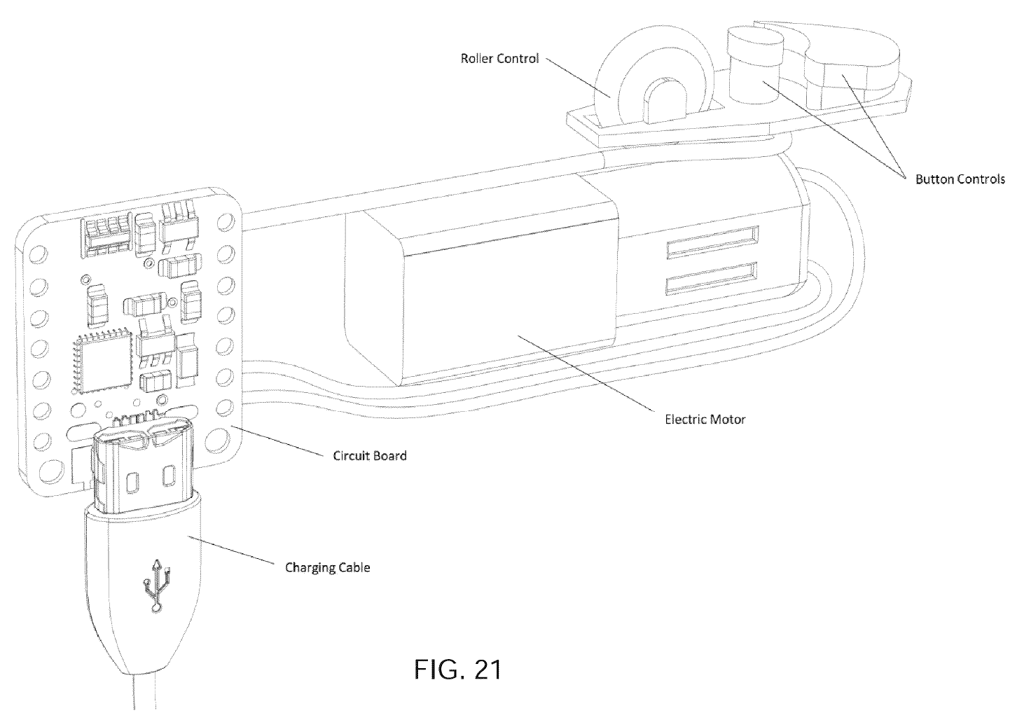

FIG. 19 is an alternative design of the device of FIG. 2 wherein the force to drive the rotor is from an electric motor. FIG. 20 is a cross-sectional perspective view of FIG. 19 and shows the internal mechanisms and electronics assembly. The motor receives power and on/off signal from the circuit board. The circuit board can be powered directly from a removable cable (external power) or can have an integrated battery as an onboard power source. The user interacts with the roller and button controls which dictate fluid output. The user can select a specific dosage per button depression or switch a continuous flow with adjustable flow rate. The control buttons send signals to the circuit board of the desired motor response. It is also possible to add a small screen or indicator next to the controls to display the mode and fluid output selected. FIG. 21 is an isolated view of only the electric motor drive components. The term “actuator” encompasses motors to operate the internal fluid pump from outside the housing and cause fluid to flow out the distal cannula, and “manual actuator” is a subset thereof.

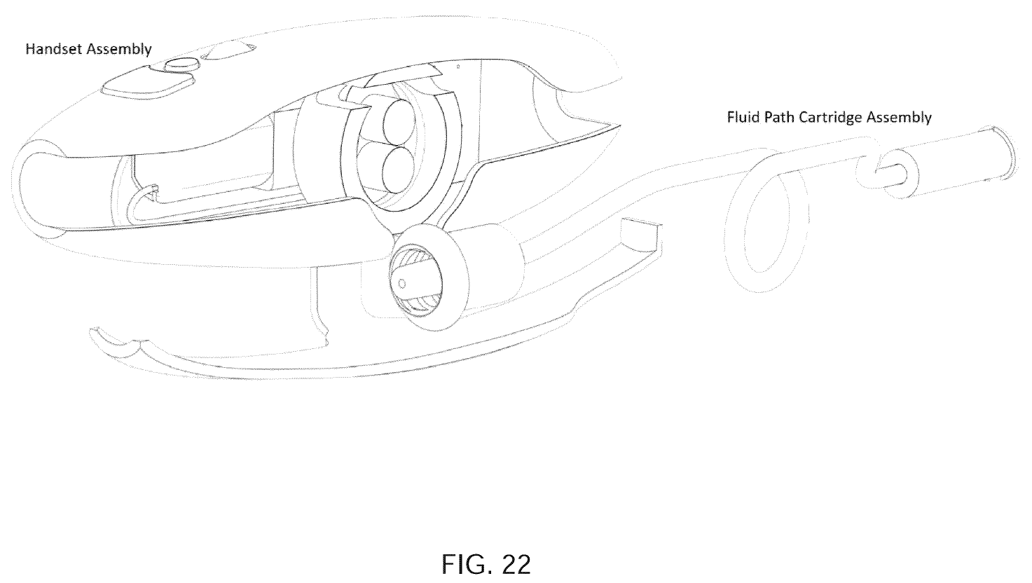

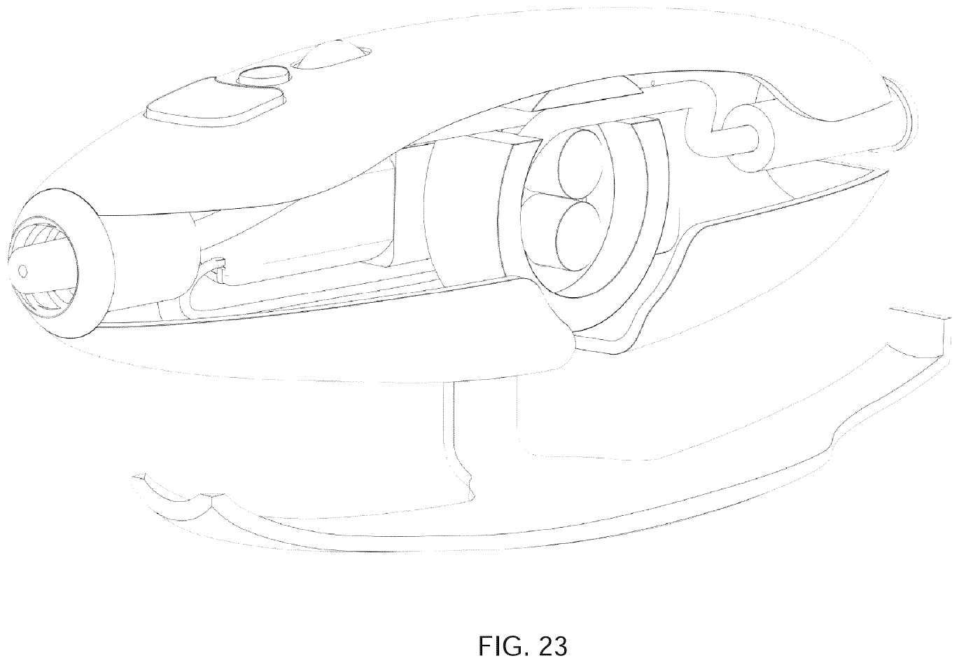

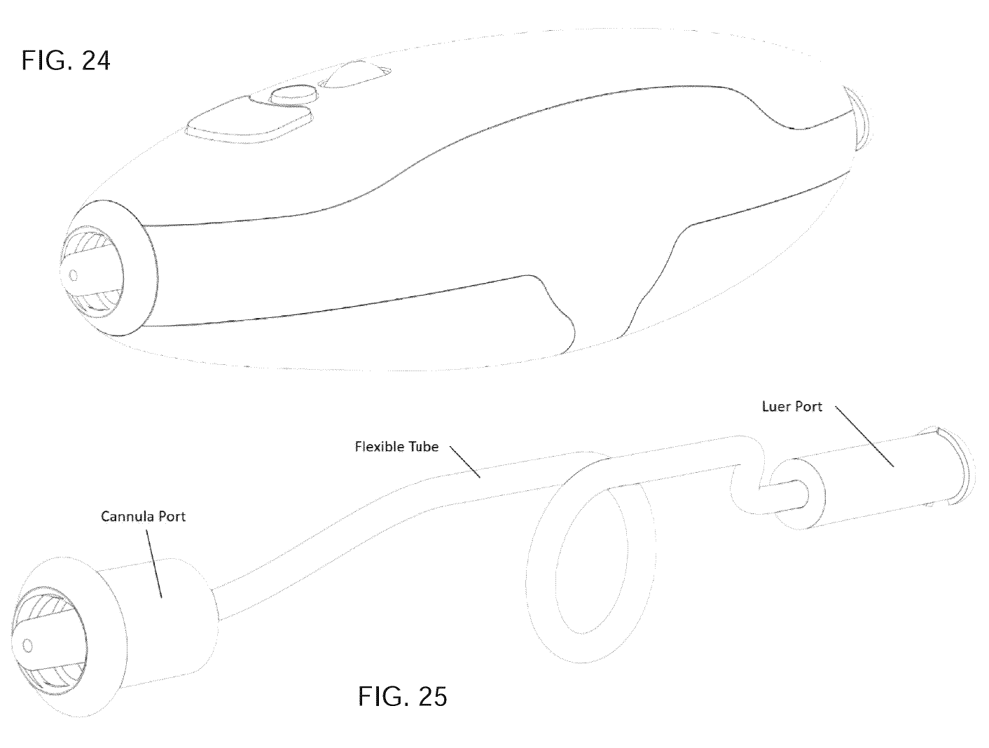

FIG. 22 shows a design in which the fluid path components are combined as a replaceable cartridge inserted within the hollow outer housing. The handset assembly can be reusable and can accommodate variations of cartridges. A door on the hollow handset assembly opens to allow the operator to install the cartridge. Once installed (FIG. 23), the door can be closed (FIG. 24). The minimum components that comprise the cartridge include: the cannula port, flexible tube and luer port as seen in FIG. 25. If internal fluid storage is a requirement, it may also include a reservoir.

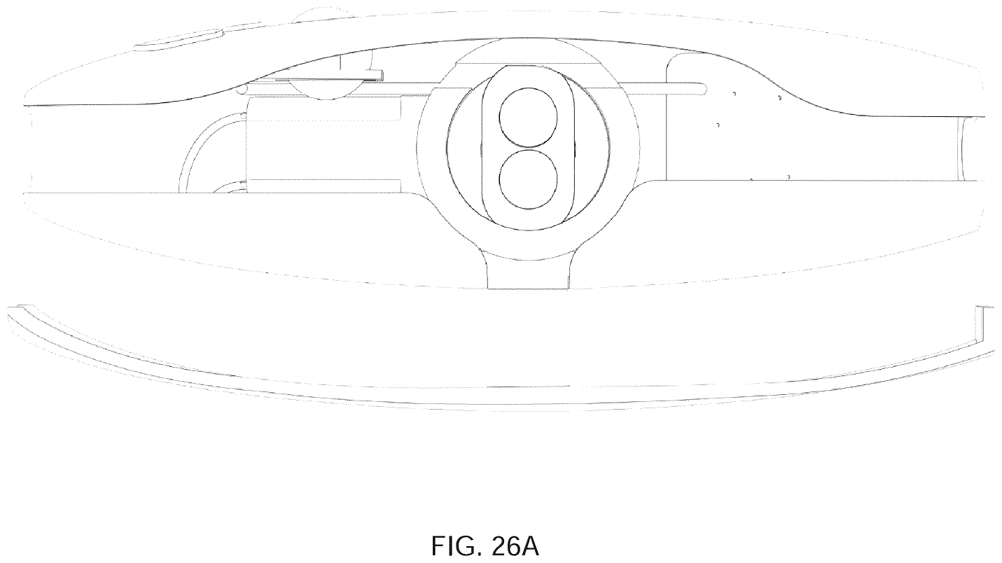

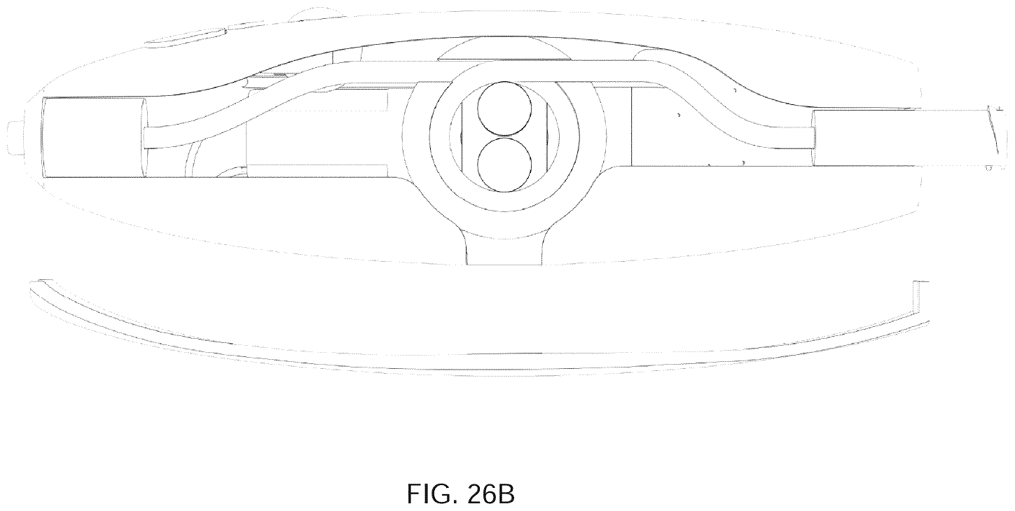

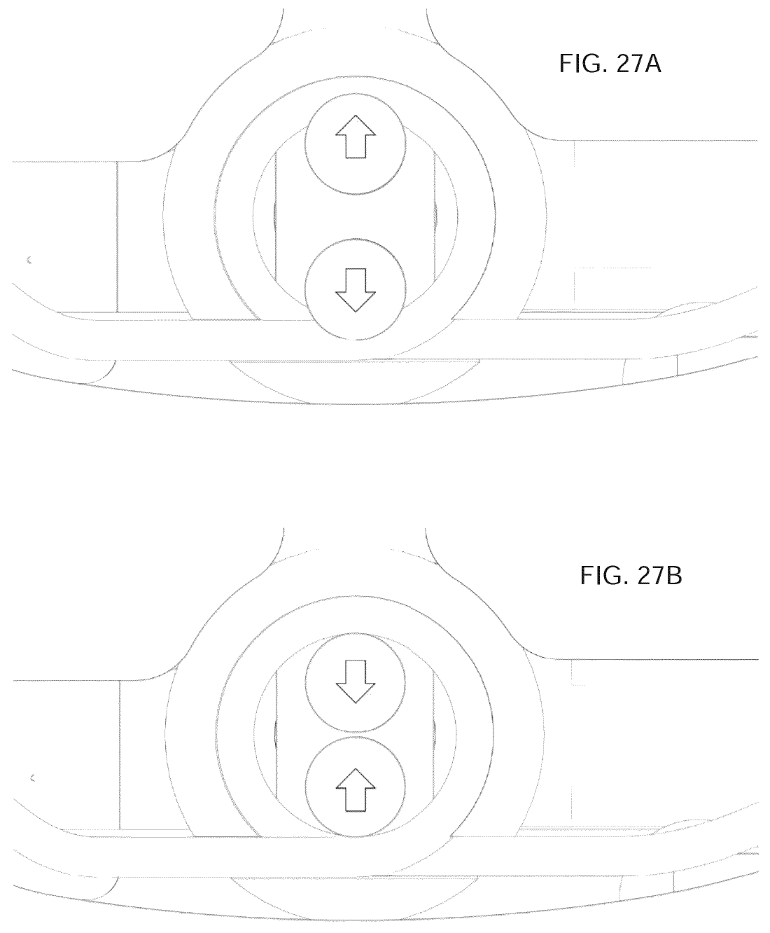

FIGS. 26A and 26B show a side view of the loading of the cartridge and the retracted rollers. For case of loading the flexible tubing between the rollers and rotor housing, the rollers can retract during loading and then be activated to engage and deform the tubing, as shown in FIG. 27A and 27B. When the tubing is installed and the rotors are still retracted, such as in FIG. 26B, fluid can be purged through the system to prepare the device.

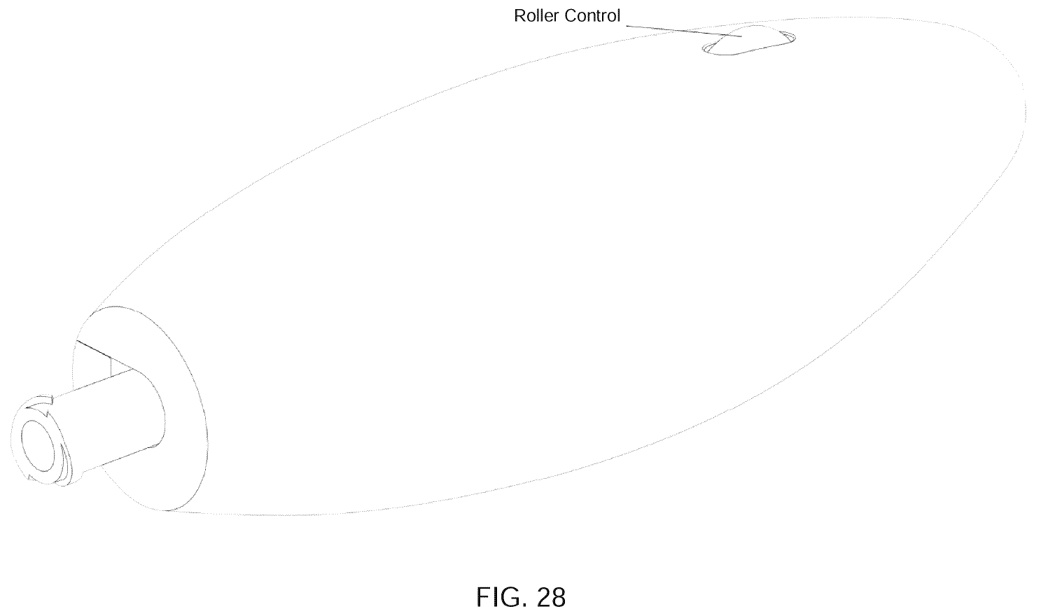

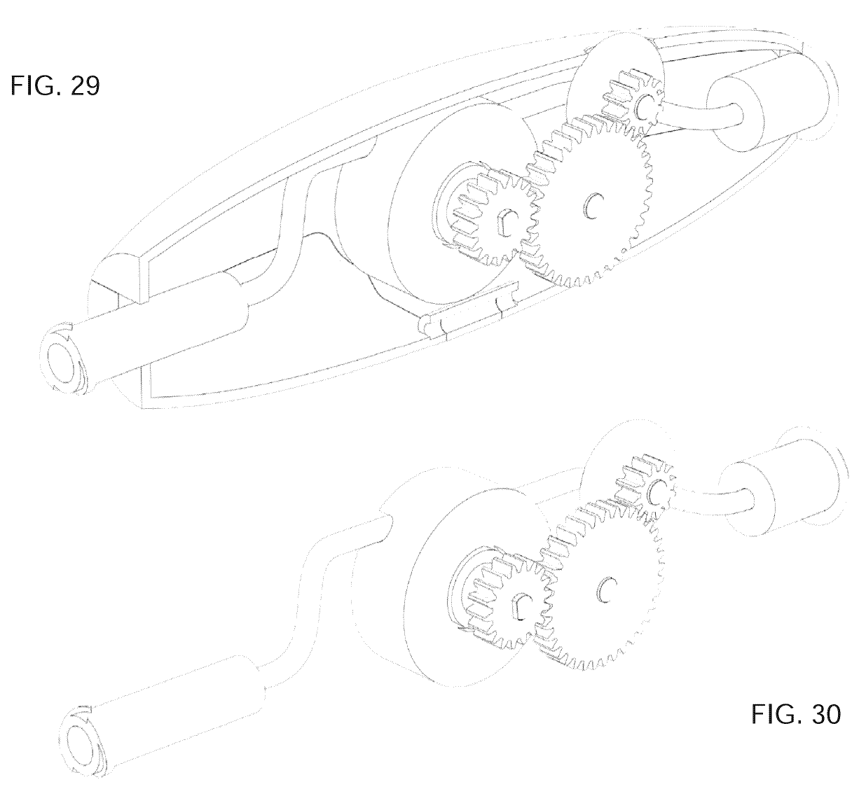

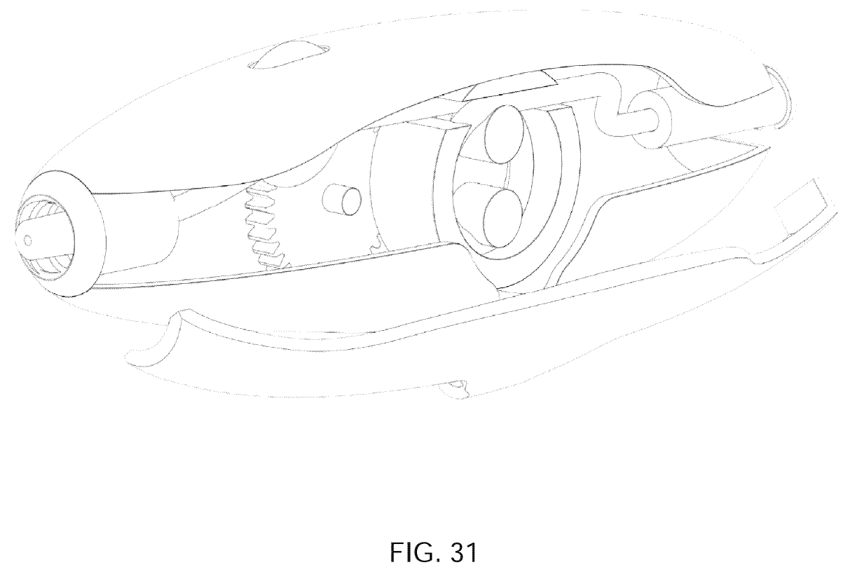

FIG. 28 is an alternative design of the device 20 of FIG. 3 wherein a lever is replaced with a roller wheel and gear train as seen in FIGS. 29 and 30. Gears can be sized to allow an increase or decrease of the rotation amount. To engage the rollers, the user would scroll the roller wheel. The roller wheel design can also be combined with a replaceable cartridge as shown in FIG. 31. Therefore, the term “manual actuator” means a manually-operated lever, roller, slider, dial, etc. that may be used to operate the internal fluid pump from outside the housing and cause fluid to flow out the distal cannula.

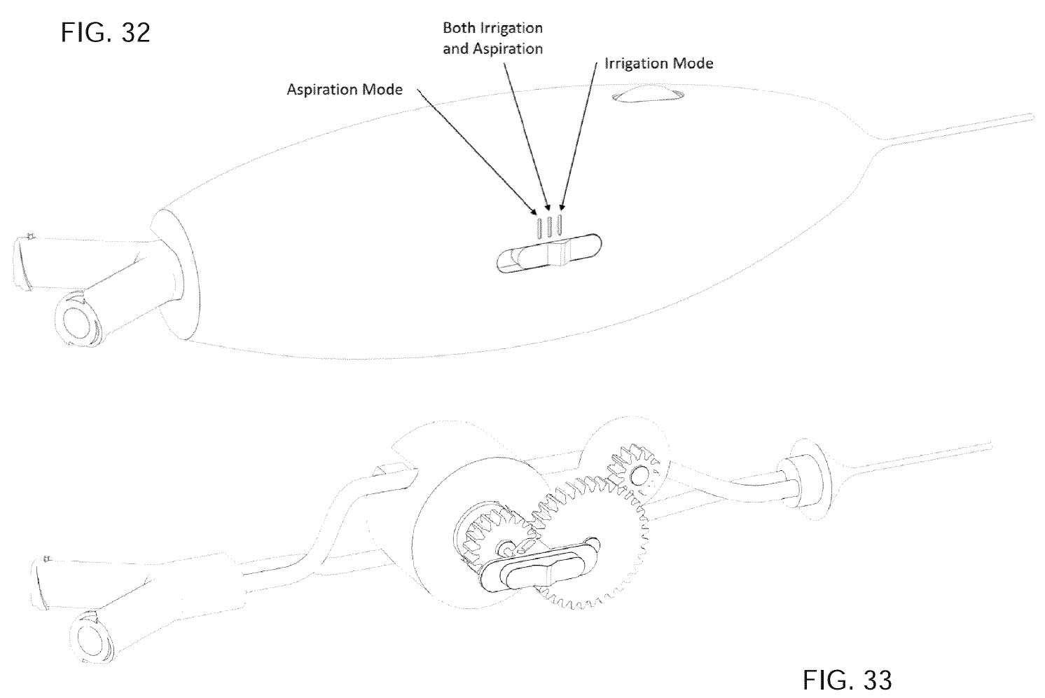

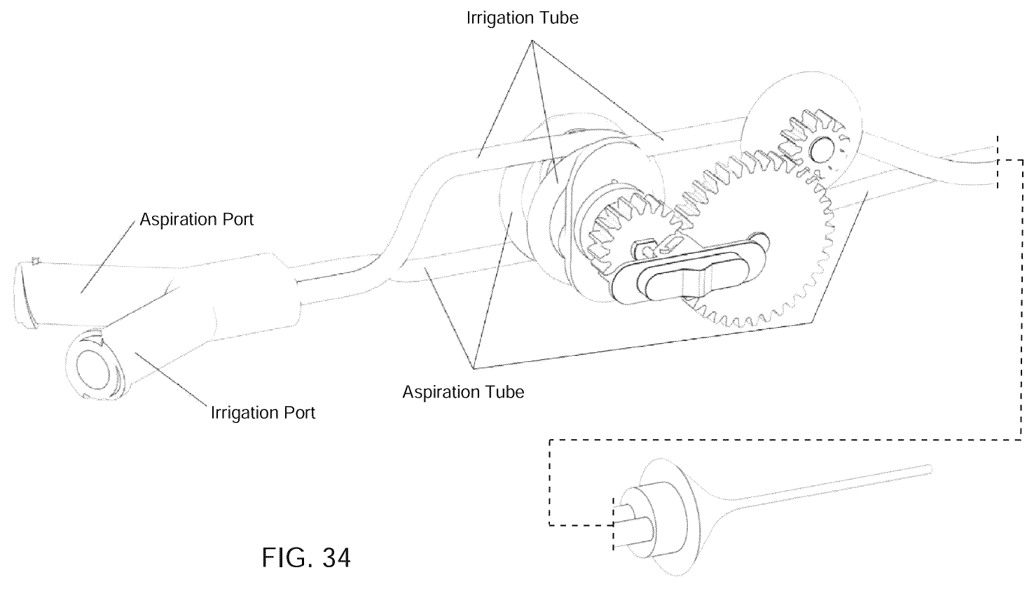

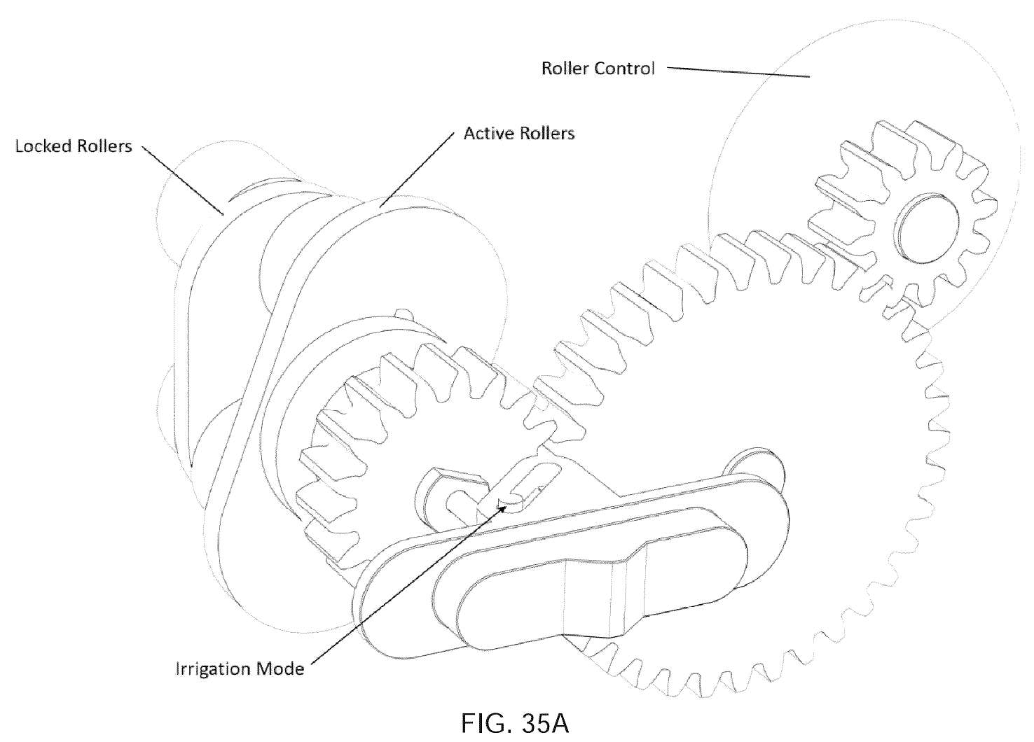

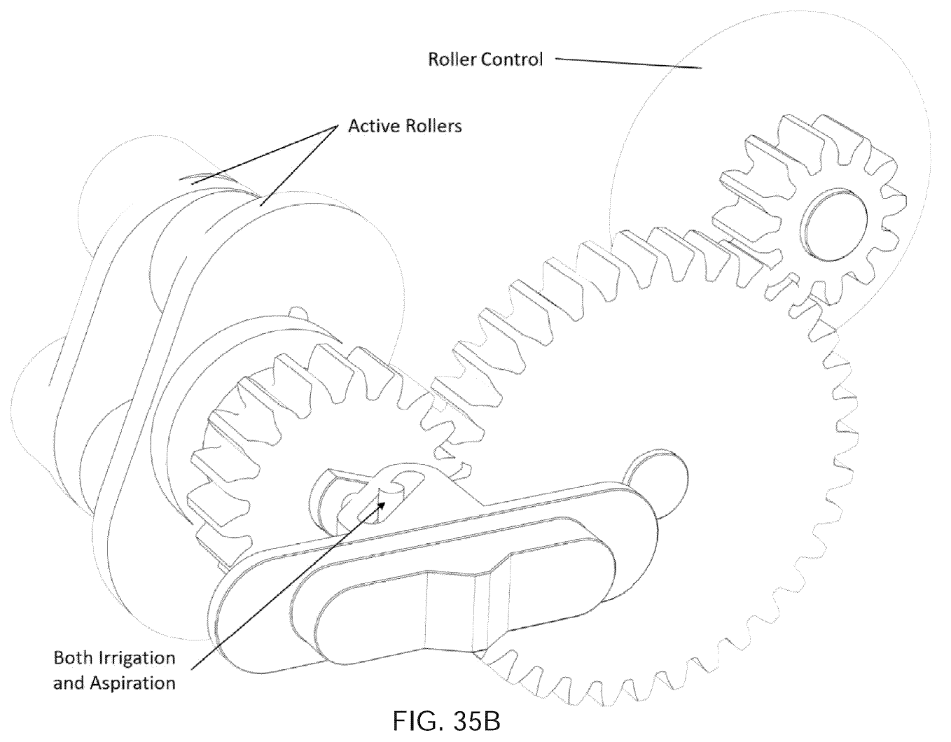

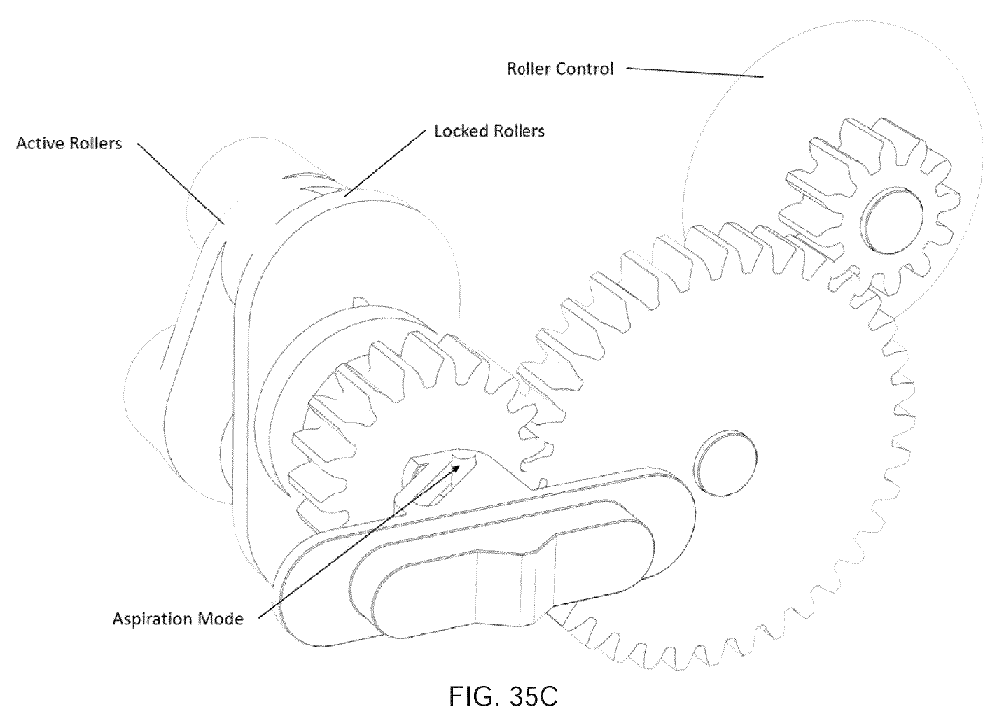

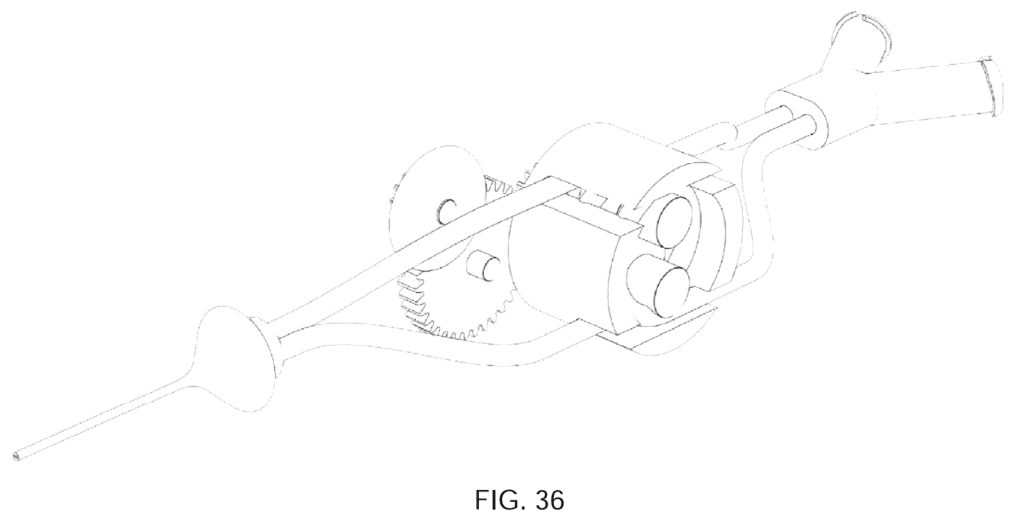

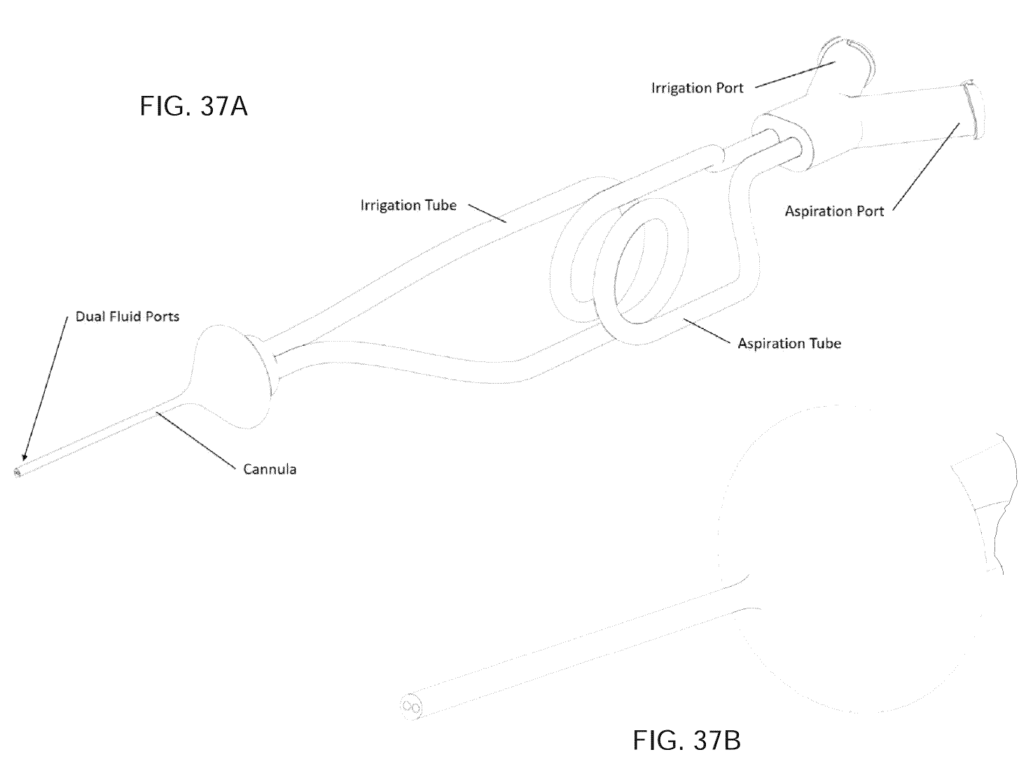

FIG. 32 shows an alternative design to the single tube cartridge described in FIG. 22. This design allows for dual fluid flow pathways in a single cartridge (FIG. 33). A selector on the side of the device seen in FIG. 32 is set by the user to engage either or both internal rotors. In the configuration shown, one tube is loaded counter clockwise, while the other is clockwise as depicted in FIG. 37A. With the tubes in opposite directions, the rotation of the rotors will cause opposite fluid flows in either tube, also referred to as irrigation and aspiration as depicted in FIG. 34. FIG. 35A-35C show how the user can move the selector to engage either or both rollers allowing for irrigation (FIG. 35A), irrigation and aspiration (FIG. 35B) or aspiration (FIG. 35C). FIG. 36 shows the extended rotor housing and slots for tube loading. The cannula will also contain dual fluid paths with dual proximal luer connectors and dual distal fluid ports for irrigation and aspiration as seen in FIG. 37B.

Aspects of the present application include:

Fluid drawn from internal or external reservoir.

Fluid enters eye through a body that penetrates the cornea/sclera and delivers fluid
a. From the distal end of body
b. From side holes of body
c. From a body that is curved to match tissue curvature
d. From a secondary fluid delivery body that is deployed from the main body (e.g. a catheter or actuated tip)
e. An application of the design would be to use the device for a canaloplasty via visco elastic fluid delivery. A primary or secondary fluid delivery body may sit against, partially inside, or fully inside the TM/SC to deliver visco elastic through the SC.
f. An alternate design is to have a detachable connection port at the end of the pump (such as a luer connection) to allow the practitioner to select an appropriate apparatus to deliver fluid. These apparatuses may include a needle, a bent or straight tube, or a specially designed part to interface with selected tissue

Fluid may be transferred by a pump that works by splitting the fluid into segments and then driving those segments forward in a rotary fashion.
a. Fluid can be transferred in a flexible tube where an external roller/wiper presses on the tube to constrict it, while advancing along the tube to move the fluid, in a peristaltic manner, or
b. Fluid can be split into chambers, where a wiper advances the fluid and a changing chamber size forces the liquid to advance out of the pump. The wiper may be a star-shaped impeller having flexible vanes, and
c. Alternatively, where the fluid path (e.g., flexible tubing or wiper assembly) can be replaced and disposed while other non-fluid path components are reusable.

The application also discloses a rotary pump mechanism configured to drive fluid in one direction with the aid of a ratchet, clutch, and one-way bearing.
a. An alternate design is to have a switch to allow the user to specify which direction the rotation/pumping is occurring.
   i. This is similar to the switch on a ratchet wrench to change direction.
   ii. In this case, the pumping rotation can turn into a vacuum to draw fluid/blood out of a specific area.
b. The design may include one or more ratchet mechanisms.
   i. Similar to tightening a bolt with a ratchet wrench, a certain amount of friction is needed to allow the ratcheting to advance. In a low friction design, a second ratcheting mechanism can be inserted to allow both ratchets to alternately lock onto a shaft and spin the shaft in one direction only.

The rotation of the pump will be controlled and mechanically driven via a manually operated arm/lever or roller wheel.
a. An alternate design is to have a mechanism (such as gears) to increase or decrease the rotation amount of torque applied versus a straight arm/lever.

The practitioner will interface with this arm/roller directly to drive the pump. The actuation of this arm/roller is to be of a size, force and movement such that it may be operable with a finger while holding the device.

The application also discloses a mechanism which allows the insertion and replacement of the fluid contacting components (i.e., fluid path).
a. Fluid path components combined as a unit can be treated as a cartridge.
   i. Individual cartridge components can vary in type, size, and function to create multiple cartridge options.
      1. Tubing diameter can vary to allow for different output volumes.
      2. The output component can be a luer to connect with another attachment or can be the intraocular fluid delivery component itself.
      3. A reservoir can be added as part of the cartridge.
   ii. Operator can select cartridge type to best suite surgical needs.
b. The handset can have a door/flap/opening that allows the user to insert the cartridge.
c. The handset can also have a mechanism to retract the rollers to allow for tubing placement and provide the option of purging fluid through the system prior to use.

The handset can be reusable while the cartridge is disposable. The application also discloses an alternate design which incorporates dual tubing paths.
a. The dual tubing components can be combined as a cartridge.
b. The dual tubes would encircle dual sets of rollers.
c. The tubes can be configured in the same or opposite direction (i.e., clockwise vs counter clockwise)
   i. If both tubes are in the same direction, both fluid flows will be in the same direction.
   ii. If tubes are wound in opposite directions, one tube will provide irrigation, and the other aspiration.
d. A selector on the side of the housing can engage either roller or both rollers to rotate with the finger wheel.
   i. User can select to irrigate, aspartate or irrigate/aspirate simultaneously.

While the invention has been described in its preferred embodiments, it is to be understood that the words which have been used are words of description and not of limitation. Therefore, changes may be made within the appended claims without departing from the true scope of the invention.

What is claimed is:

1. A hand-operated manual intraocular fluid delivery device, comprising:
a handle having a hollow outer housing and a chamber located within the housing, the housing having a distal end with a fluid outlet and a proximal end with a fluid inlet;
flexible inlet tubing connected to the fluid inlet and extending to the chamber and flexible outlet tubing connected to the fluid outlet and extending to the chamber;
a cannula having a distal tip adapted for insertion into an intraocular space in an eye and a proximal end in fluid communication with the fluid outlet;
a manual actuator mounted in the housing and being accessible from an exterior thereof; and
a rotor arranged to rotate within the chamber and configured to be rotated by the manual actuator, wherein
manually operating the manual actuator causes the rotor to advance a measure of fluid from the inlet tubing through the chamber to the outlet tubing, and from there through the fluid outlet and through the cannula distal tip into the intraocular space.

2. The device of claim 1, wherein the inlet tubing and outlet tubing are continuous and pass through the chamber and the rotor compresses the tubing within the chamber to create peristaltic fluid motion.

3. The device of claim 2, wherein the rotor includes a plurality of rollers mounted for rotation within the chamber each of which compresses the tubing against an inner wall of the chamber.

4. The device of claim 1, wherein the chamber is sealed and fluid-filled, and the rotor directly contacts the fluid and impels the fluid to move through the chamber to the outlet tubing.

5. The device of claim 4, wherein the rotor has a wiper that contacts an inner wall of the chamber.

6. The device of claim 5, wherein the wiper comprises a pair of solid wipers that are aligned within a linear channel in an off-center hub assembly that rotates within the chamber, the wipers being biased apart by an inner spring.

7. The device of claim 5, wherein the wiper comprises a number of flexible spokes or vanes of the same length projecting outward from an off-center hub that rotates within the chamber.

8. The device of claim 1, wherein the cannula is detachable from the fluid outlet with threading, a bayonet lock or a luer lock.

9. The device of claim 1, further including an internal fluid reservoir within and connected to a luer fitting on a proximal end of the housing to which a source may attach to supply fluid to the reservoir.

10. The device of claim 1, wherein the housing is between 2-8 inches long and between 0.4-2.5 inches in diameter.

11. The device of claim 1, further including a gear train between the manual actuator and the rotor.

12. The device of claim 1, wherein the device includes dual fluid flow pathways for selective irrigation and aspiration, and an external selector to switch therebetween.

13. The device of claim 12, wherein the inlet tubing and outlet tubing are continuous and pass through the chamber and the rotor compresses the tubing within the chamber to create peristaltic fluid motion, and there are two lengths of tubing passing through the chamber-a first one coiled counter-clockwise and a second one coiled clockwise-such that the rotation of the rotors will cause opposite fluid flows in either tube.

14. The device of claim 13, wherein the cannula contain dual fluid paths, and the device has dual proximal luer connectors and dual distal fluid ports for irrigation and aspiration.

15. An intraocular fluid delivery device, comprising:
a handle having a hollow outer housing and a chamber located within the housing;
an actuator mounted to the housing and being accessible from an exterior thereof;
a rotor arranged to rotate within the chamber and configured to be rotated by the actuator;
a cartridge removably mounted within the housing, the cartridge including a distal fluid outlet and a proximal fluid inlet with flexible tubing therebetween, the chamber defining a pathway therethrough for the flexible tubing and the fluid outlet being located at a distal end of the housing; and
a cannula having a distal tip adapted for insertion into an intraocular space in an eye and a proximal end in fluid communication with the fluid outlet, wherein
operating the actuator causes the rotor to advance a measure of fluid through the flexible tubing, and from there through the fluid outlet and through the cannula distal tip into the intraocular space.

16. The device of claim 15, wherein the rotor comprises rollers that compress the flexible tubing within the chamber to create peristaltic fluid motion.

17. The device of claim 15, wherein the cannula is detachable from the fluid outlet with threading, a bayonet lock or a luer lock.

18. The device of claim 15, wherein the actuator is a manually-operated button, wheel, lever, roller, slider or dial.

19. The device of claim 15, wherein the actuator includes a motor.

20. The device of claim 15, wherein the cartridge further includes a fluid reservoir connected to the fluid inlet such that a source may supply fluid to the reservoir.
